(19) United States
(12) Patent Application Publication
(10) Pub. No.: US 2021/0269797 A1
(43) Pub. Date: Sep. 2, 2021

(54) METHODS AND COMPOSITIONS FOR TARGETING PD-L1

(71) Applicant: ALIGOS THERAPEUTICS, INC., South San Francisco, CA (US)

(72) Inventors: Leonid Beigelman, San Francisco, CA (US); Megan Elizabeth Fitzgerald, San Francisco, CA (US); Saul Martinez Montero, San Francisco, CA (US); Aneerban Bhattacharya, San Mateo, CA (US)

(21) Appl. No.: 17/249,335

(22) Filed: Feb. 26, 2021

Related U.S. Application Data
(60) Provisional application No. 62/983,147, filed on Feb. 28, 2020.

(51) Int. Cl. C12N 15/113; A61K 45/06

(52) U.S. Cl. CPC C12N 15/113; A61K 45/06; C12N 2310/11; C12N 2310/346; C12N 2310/3231; C12N 2310/3515; C12N 2310/321; C12N 2310/322; C12N 2310/33

(57) ABSTRACT

The present disclosure relates to antisense oligonucleotides (ASOs) directed to mRNA transcripts of CD274 to cause downregulation of programmed death-ligand 1 (PD-L1) expression in humans. The ASO can be constructed of unmodified nucleotides or modified nucleotides that exhibit modified sugars, nucleobases, linkages, or covalently bound targeting moieties. Also disclosed herein are pharmaceutical compositions of ASOs and uses of or methods of using the ASOs for the treatment of PD-L1 related diseases including but not limited to liver diseases, cancer, hepatocellular carcinoma, viral diseases, or hepatitis B.

### INCORPORATION BY REFERENCE TO PRIORITY APPLICATION

This application claims priority to U.S. Provisional Application Ser. No. 62/983,147, filed Feb. 28, 2020, which is hereby incorporated herein by reference in its entirety.

### FIELD

The present application relates to the fields of chemistry, biochemistry, molecular biology and medicine. The present disclosure relates to antisense oligonucleotides (ASOs) directed to mRNA transcripts of CD274 to cause downregulation of programmed death-ligand 1 (PD-L1) expression in humans. The ASO can be constructed of unmodified nucleotides or modified nucleotides that exhibit modified sugars, nucleobases, linkages, or covalently bound targeting and/or lipophilic moieties. Also disclosed herein are pharmaceutical compositions of ASOs and uses of or methods of using the ASOs for the treatment of PD-L1 related diseases including but not limited to liver diseases, cancer, hepatocellular carcinoma, viral diseases, or hepatitis B.

### BACKGROUND

The programmed cell death 1 (PD-1) immune checkpoint expressed on the surface of activated CD4⁺ and CD8⁺ T cells controls an inhibitory mechanism to prevent autoimmunity. Engagement of PD-1 by programmed death-ligand 1 (PD-L1) expressed on the multitude of cell types, including macrophages, dendritic cells, mast cells as well as non-hematopoietic cells, induces T cell exhaustion resulting in reduction or loss of effector cytokine production (e.g. IL-2, TNF-α, IFN-γ) and upregulation of other inhibitory receptors and immune checkpoints (e.g. CTLA-4, LAG-3, and BTLA), or T cell apoptosis. High expression of PD-L1 is exhibited by many types of cancers to escape tumor immune surveillance and has been associated with poorer prognosis. PD-1-mediated immunosuppression is also linked to some viral infections, such as hepatitis B. There is an ongoing need for PD-1/PD-L1 therapies and improvements thereof for the treatment of disease.

### SUMMARY

Embodiments provided herein related to antisense oligonucleotides (ASOs) that target to CD274, compositions thereof, and uses thereof for the treatment, inhibition, amelioration, prevention or slowing of diseases or conditions associated with PD-L1 dysregulation.

Some embodiments provided herein relate to antisense oligonucleotides (ASOs) that target human CD274 mRNA. In some embodiments, the ASO comprises 14 to 20 nucleotides selected from the group consisting of unmodified nucleotides and modified nucleosides. In some embodiments, each of the modified nucleosides contains a modified sugar, contains a modified nucleobase or is abasic, or both contains a modified sugar and contains a modified nucleobase or is abasic. In some embodiments, each linkage between the nucleosides is a phosphorothioate, phosphodiester, phosphoramidate, thiophosphoramidate, methylphosphate, methylphosphonate, boranophosphate or any combination thereof. In some embodiments, the ASO is at least 85% complementary to a fragment of human CD274 mRNA. In some embodiments, where the ASO is 14 or 15 nucleotides in length, the ASO includes no nucleotide mismatches to the fragment of human CD274 mRNA. In some embodiments, where the ASO is 16 or 17 nucleotides in length, the ASO includes zero or one nucleotide mismatches to the fragment of human CD274 mRNA, wherein the mismatches occur in the flank regions within the first three or last three nucleotides. In some embodiments, where the ASO is 18 or 19 nucleotides in length, the ASO includes zero, one, or two nucleotide mismatches to the fragment of human CD274 mRNA, wherein the mismatches occur in the flank regions within the first four or last four nucleotides. In some embodiments, where the ASO is 20 nucleotides in length, the ASO includes zero, one, or two nucleotide mismatches to the fragment of human CD274 mRNA, wherein the mismatches occur in the flank regions within the first five or last five nucleotides. In some embodiments, the ASO has a sequence as set forth in any one of SEQ ID NOs: 2-301. In some embodiments, the ASO is 14, 15, 16, 17, 18, 19, or 20 nucleotides in length. In some embodiments, the modified sugar is selected from the group consisting of 2′-OMe, 2′-F, 2′-MOE, 2′-araF, 2′-araOH, 2′-OEt, 2′-O-alkyl, LNA, scpBNA, AmNA, cEt, and ENA. In some embodiments, the modified nucleobase is selected from the group consisting of 5-OH—C, 2S-T, 8-NH2-A, 8-NH2-G, and 5-methyl-C. In some embodiments, the ASO further includes a targeting moiety. In some embodiments, the targeting moiety is conjugated to the ASO at the 5′ end, 3′ end, or both. In some embodiments, the targeting moiety is GalNAc, folic acid, cholesterol, tocopherol, or palmitate. In some embodiments, the ASO is a gapmer, mixmer, or blockmer. In some embodiments, the base of the ASO is selected from the group consisting of adenine, guanine, cytosine, thymine, uracil, pseudouracil, 2-thio-uracil, dihydrouracil, 5-bromo-uracil, 5-iodo-uracil, 5′-methyl-cytosine, 7-deazapurine, 2,6-diaminopurine, inosine, phenoxazine, and

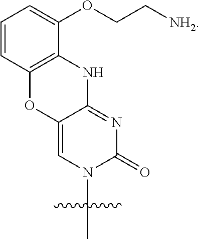 [SMILES: *n1cc2c(nc1=O)Nc1c(OCCN)cccc1O2]

In some embodiments, the modified nucleobase is selected from the group consisting of pseudouracil, 2-thio-uracil, dihydrouracil, 5-bromo-uracil, 5-iodo-uracil, 5′-methyl-cytosine, 7-deazapurine, 2,6-diaminopurine, inosine, phenoxazine, and

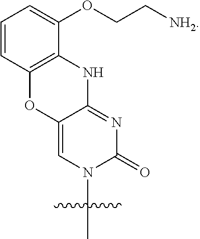 [SMILES: *n1cc2c(nc1=O)Nc1c(OCCN)cccc1O2]

Some embodiments provided herein relate to pharmaceutical compositions that include an effective amount of any ASO as described herein. In some embodiments, the pharmaceutical composition includes a pharmaceutically acceptable carrier, diluent, excipient, or combination thereof.

Some embodiments provided herein relate to any ASO as described herein or any pharmaceutical composition as described herein for use in treating a disorder or disease, such as an infection or a cancer, such as for use in treating hepatitis B or for use in treating hepatocellular carcinoma (HCC). In some embodiments, the ASO is used in combination with surgery, radiation therapy, chemotherapy, targeted therapy, immunotherapy, hormonal therapy, or antiviral therapy. In some embodiments, the ASO comprises an ASO against PD-L1 and an ASO or an siRNA against hepatitis B virus (HBV).

Some embodiments provided herein relate to methods for treating a disease or disorder in a subject. In some embodiments, the methods include administering to the subject an effective amount of any ASO as described herein or an effective amount of any pharmaceutical composition as described herein. In some embodiments, the disease or disorder is an infection or a cancer, such as hepatitis B or hepatocellular carcinoma. In some embodiments, the methods further include administering surgery, radiation therapy, chemotherapy, targeted therapy, immunotherapy, hormonal therapy, or antiviral therapy.

Additional embodiments are described in greater detail below.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments, and are not intended to be limiting in scope.

FIG. 1 depicts percent PD-L1 knockdown in human hepatocellular carcinoma cells (SNU-387 cells) using an exemplary anti-sense oligonucleotide as described herein.

FIG. 2 depicts the fraction of PD-L1 mRNA remaining after treatment of SNU-387 cells with exemplary anti-sense oligonucleotides as described herein.

### DETAILED DESCRIPTION

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The practice of the present disclosure will employ, unless indicated specifically to the contrary, conventional methods of molecular biology and recombinant DNA techniques within the skill of the art.

Hepatocellular carcinoma (HCC) is the most common form of liver cancer. HCC can be caused by a variety of conditions, such as alcohol consumption, cirrhosis, and viral infections that cause hepatitis, such as hepatitis B virus, hepatitis C virus, and hepatitis D virus. The inflammation, fibrosis, and cirrhosis linked with these conditions can induce malignancies in affected liver cells. HCC has relatively poor prognosis, with a five-year survival rate of about 30%, depending on if full surgical resection of the tumor is possible.

For early disease, surgical resection is used. However, most HCC are identified at later stages because of difficulties in diagnosing. Upon late stage diagnosis, the tumors are unresectable and most patients are given systemic therapies. The current standard of care in front line are multi-kinase inhibitors (including, for example, sorafenib and/or lenvatinib). Most patients are refractory or relapse from these treatments, and undergo second line therapies that have anti-angiogenic agents (including, for example, Regorafinib, Cabozantinib, and/or Ramicirumab) or immune checkpoint inhibitors (including, for example, nibolumab and/or pembrolizumab). However, most patients do not respond to first and second therapies, and the clinical benefit is poor, with overall survival not exceeding one year. In addition, biomarker driven therapies are lacking. Thus, there is a need to develop more tolerable and efficacious therapies for the treatment of HCC and related liver disorders.

HBV is a partially double-stranded circular DNA of about 3.2 kilobase (kb) pairs, and is classified into eight genotypes, A to H. The HBV replication pathway has been studied in great detail. One part of replication includes the formation of the covalently closed circular DNA (cccDNA) form. The presence of the cccDNA gives rise to the risk of viral reemergence throughout the life of the host organism. HBV carriers can transmit the disease for many years. An estimated 300 million people are living with hepatitis B virus infection, and it is estimated that over 750,000 people worldwide die of hepatitis B each year. In addition, immunosuppressed individuals or individuals undergoing chemotherapy are especially at risk for reactivation of an HBV infection. HBV can be acute and/or chronic. Acute HBV infection can be either asymptomatic or present with symptomatic acute hepatitis.

HBV can be transmitted by blood, semen, and/or another body fluid. This can occur through direct blood-to-blood contact, unprotected sex, sharing of needles, and from an infected mother to her baby during the delivery process. The HBV surface antigen (HBsAg) is most frequently used to screen for the presence of this infection. Currently available medications do not cure HBV and/or HDV infection. Rather, the medications suppress replication of the virus.

The hepatitis D virus (HDV) is a DNA virus, also in the Hepadnaviridae family of viruses. HDV can propagate only in the presence of HBV. The routes of transmission of HDV are similar to those for HBV. Transmission of HDV can occur either via simultaneous infection with HBV (coinfection) or in addition to chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B, hepatitis D has the highest fatality rate of all the hepatitis infections, at 20%. There is currently no cure or vaccine for hepatitis D.

Programmed cell death 1, or programmed death 1 (PD-1) is a 268 amino acid long type I transmembrane protein found as a surface marker on T cells and other immune cells. As an immune checkpoint, PD-1 serves to negatively regulate immune responses to prevent autoimmune disorder. PD-1 protein (NCBI accession number NP 005009.2) is expressed from the cluster of differentiation 279 (CD279) gene (NCBI accession number NG 012110.1) or mRNA transcript (NCBI accession number NM_005018.3). In some preferred embodiments, PD-1 is the human PD-1 protein, and CD279 is the human CD279 transcript or gene on chromosome 2. It should be understood that a person with ordinary skill in the art would view the terms PD-1 and CD279 as often nominally interchangeable when considering the nucleic acid (DNA or RNA) or corresponding translated protein, or the sequences thereof.

Programmed cell death-ligand 1, or programmed death-ligand 1 (PD-L1), also known as B7 homolog 1 (B7-H1) is 272 amino acid long type I transmembrane protein found as a surface marker on many different cell types. PD-L1 is a major ligand of PD-1 and results in inhibition of T cell cytotoxicity and cytokine production. Cancer cells such as HCC cells take advantage of this immune checkpoint by upregulating PD-L1 expression, resulting in dysfunctional anti-tumor immunity by proximal T cells. Viruses also have been observed to modulate the PD-1/PD-L1 pathway to improve infectivity. Hepatitis B virus has been shown to upregulate PD-L1 in infected hepatocytes, and PD-1 in associated T cells. PD-L1 protein (NCBI accession number NP_054862.1) is expressed from the cluster of differentiation 274 (CD274) transcript (NCBI accession number NM_014143.4). In some preferred embodiments, PD-L1 is the human PD-L1 protein, and CD274 is the human CD274 transcript or gene on chromosome 9. It should be understood that a person with ordinary skill in the art would view the terms PD-L1 and CD274 as often nominally interchangeable when considering the nucleic acid (DNA or RNA) or corresponding translated protein, or the sequences thereof.

As used herein, an “oligonucleotide” refers to a single stranded nucleic acid molecule that includes unmodified nucleotides, modified nucleotides or a combination of modified nucleotides and unmodified nucleotides.

As used herein, an “unmodified nucleotide” is a nucleotide that has a deoxyribose sugar or a ribose sugar and a nucleobase selected from adenine, cytosine, guanine, thymine and uracil. An unmodified nucleotide can also be considered to have a nucleoside selected from cytidine, uridine, 5-methyluridine, guanosine, adenosine, deoxycytidine, deoxyuridine, deoxyguanosine, deoxyadenosine, and thymidine. The structures of deoxyribose, ribose, adenine, cytosine, guanine, thymine, uracil, cytidine, uridine, 5-methyluridine, guanosine, adenosine, deoxycytidine, deoxyuridine, deoxyguanosine, deoxyadenosine, and thymidine are known to those skilled in the art.

As used herein, a “deoxyribose sugar” has the structure

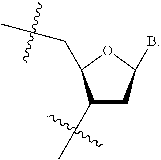

B indicates a nucleobase.

As used herein, a “ribose sugar” has the structure

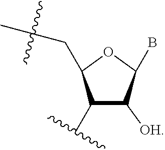

B indicates a nucleobase.

Relevant positions of the 5-membered sugar ring is provided:

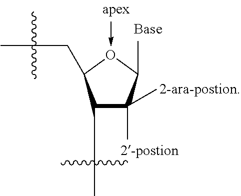

As used herein, “modified nucleotide” refers to a nucleotide that (a) includes or contains a modified sugar, (b) includes or contains a modified base or is abasic, or (c) both (a) includes or contains a modified deoxyribose and (b) includes or contains a modified base or is abasic. A modified sugar refers to either a modified deoxyribose sugar or modified ribose sugar.

As used herein, the term “modified deoxyribose” refers to a deoxyribose sugar that is substituted at one or more positions with a non-hydrogen substituent. The modifications on the deoxy sugar ring can be at any position of the ring, including at the 2′-carbon. As used herein, the term “modified ribose sugar” refers to a ribose sugar that is substituted at one or more positions with a non-hydrogen substituent. The modifications on the deoxyribose sugar or ribose sugar can be at any position of the ring, including at the 2′-carbon.

Examples of modified sugars include but are not limited to 2′-deoxy-2′-fluoro ribose (2′-F), 2′-deoxy-2′-fluoro-arabinonucloetide (2′-araF), 2′-O-methyl ribose (2′-OMe), 2′-O-(2-methoxyethyl) ribose (2′-MOE), locked nucleic acid (LNA), 2′-O-ethyl ribose (2′-OEt), 2′-O, 2′-O-alkyl, (S)-constrained ethyl (cEt), ethylene-bridged nucleic acid (ENA), 4′-C-spirocyclopropylene bridged nucleic acid (scpBNA), amido-bridged nucleic acid (AmNA), unlocked nucleic acid (UNA).

As used herein, “2′-F” refers to a modified deoxyribose sugar that has 2′ fluorine substitution and has the structure

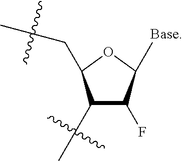

As used herein, “2′-araF” refers to a modified ribose sugar that has a fluorine group attached to 2′ position, and has the structure

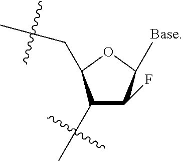

As used herein, “2′-araOH” refers to a modified ribose sugar that has a hydroxy group attached to 2′ position, and has the structure

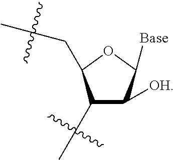

As used herein, “2′-OMe” refers to a modified ribose sugar that has a methyl group attached to the 2′ hydroxyl and has the structure

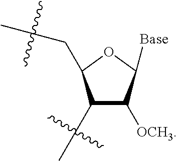

As used herein, “2′-MOE” refers to a modified ribose sugar that has a 2-methoxyethyl group attached to the 2′ hydroxyl and has the structure

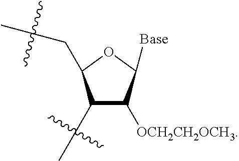

As used herein, a “locked nucleic acid” or “LNA” refers to a modified ribose sugar that includes a linkage that connects the 2′-position to the 4′-position of the 5-membered ring. Examples of locked nucleic acids include

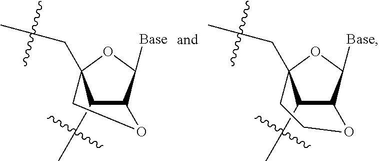

and those described in PCT publications WO 2011/052436, WO 2014/046212, and WO 2015/125783, each of which are hereby expressly incorporated by reference for the purpose of their disclosure of LNAs.

As used herein, “2′-O-Ethyl” refers to a modified ribose sugar that has an ethyl group attached to the 2′ hydroxyl and has the structure

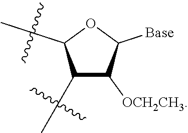

As used herein, “cEt” refers to a modified ribose sugar that includes a methyl that bridges the 2′ hydroxyl and the 4′ carbon, and has the structure

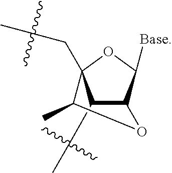

As used herein, “scpBNA” refers to a modified ribose sugar where a cyclopropane bridges the 2′ hydroxyl and 4′ carbon and has the structure

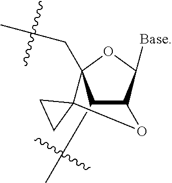

As used herein, “AmNA” refers to a modified ribose sugar where the 2′ and 4′ carbon are bridged with an amide bond and has the structure

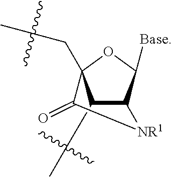

As used herein, an “unlocked nucleic acid” or “UNA” refers to a modified nucleotide wherein the bond between the 2′-position and the 3′-position of the 5-membered sugar ring is not present (acyclic ribose), and has the structure

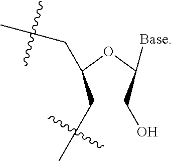

In each of the structures, the “Base”, referring to a nucleobase, can be an unmodified base, a modified base or absent, such that the nucleotide is abasic. When not indicated, the nucleotide may be an unmodified nucleotide, modified nucleotide, or abasic.

A “modified base” refers to any base other than adenine, cytosine, guanine, thymine and uracil. For example, a modified base can be a substituted adenine, a substituted cytosine, a substituted 5-methylcytosine, a substituted guanine, a substituted thymine, or a substituted uracil. Alternatively, a modified base can make up a modified nucleoside such as a substituted cytidine, a substituted 5-methyl-cytidine, a substituted uridine, a substituted 5-methyluridine, a substituted guanosine, a substituted adenosine, a substituted deoxycytidine, a substituted 5-methyl-deoxycytidine, a substituted deoxyuridine, a substituted deoxyguanosine, a substituted deoxyadenosine, or a substituted thymidine. The modified base can be monocyclic, bicyclic or tricyclic. A non-limiting list of modified bases include hypoxanthine, xanthine, 7-methylguanine, 5,6-dihydrouracil, 5-methyl-cytosine, 5-hydroxymethylcytosine, pseudouracil, 2-thio-uracil, dihydrouracil, 5-bromo-uracil, 5-iodo-uracil, 7-deazapurine, 2,6-diaminopurine, inosine, phenoxazine, and

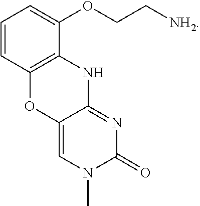 [SMILES: Cn1cc2c(nc1=O)Nc1c(OCCN)cccc1O2]

Examples of modified nucleotides include but are not limited to 5-hydroxy-deoxycytosine (5-OH-dC), 2-thio-deoxythymine (2S-T), 8-amino-deoxyguanine (8-NH2-dG), and 8-amino-deoxyadenosine (8-NH2-dA). Any of these modified nucleotides can be found in the ribonucleic acid/ribose form. For example, 5-hydroxy-cytosine, 2-thio-uracil, 8-amino-guanine, and 8-amino-adenosine.

As used herein, “5-OH-dC” has the structure

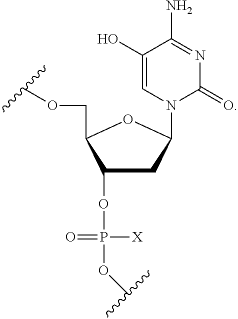

As used herein, “2S-T” has the structure

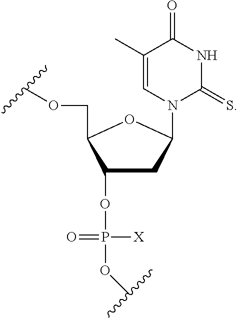

As used herein, “8-NH2-dG” has the structure

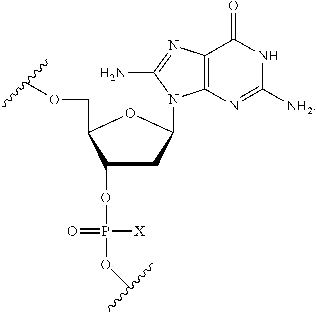

As used herein, “8-NH2-dA” has the structure

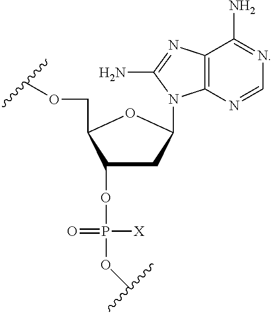

In the preceding structures, X is an oxygen, sulfur, methyl, or acetate group.

As used herein, “5-hydroxy-cytosine” is a nucleobase that may be attached to a ribose to form a nucleotide, and has the structure

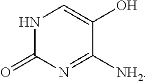

As used herein, “2-thio-uracil” is a nucleobase that may be attached to a ribose to form a nucleotide, and has the structure

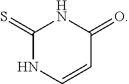

As used herein, “8-amino-guanine” is a nucleobase that may be attached to a ribose to form a nucleotide, and has the structure

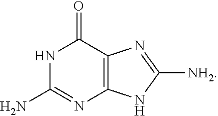 [SMILES: Nc1nc2[nH]c(N)nc2c(=O)[nH]1]

As used herein, “8-amino-adenosine” is a nucleobase that may be attached to a ribose to form a nucleotide, and has the structure

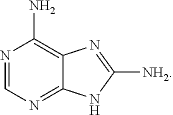 [SMILES: Nc1nc2c(N)ncnc2[nH]1]

When a specific linkage between the nucleosides are not specified, the linkage may be a phosphodiester or a non-phosphodiester linkage, such as a phosphorothioate, a methylphosphonate, a phosphoramidate, a thiophosphoramidate, a boranophosphate, or a phosophonoacetate. The phosphodiester can have the structure

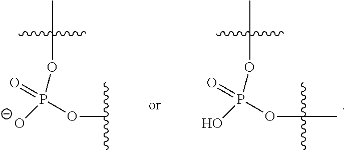 [SMILES: *OP(=O)([O-])O* ; *OP(=O)(O)O*]

As used herein, a phosphorothioate is used as understood by those skilled in the art and refers to a phosphate wherein one oxygen is replaced with a sulfur. The phosphorothioate can have the structure

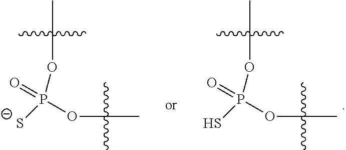 [SMILES: *OP(=O)([S-])O* ; *OP(=O)(S)O*]

As used herein, a methylphosphonate is used as understood by those skilled in the art and refers to a phosphate wherein one oxygen is replaced with a methyl. The methylphosphonate can have the structure

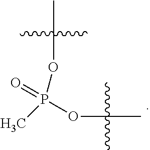 [SMILES: *OP(C)(=O)O*]

As used herein, a phosphoramidate is used as understood by those skilled in the art and refers to a phosphate wherein one oxygen is replaced with an amide. The phosphoramidate can have the structure

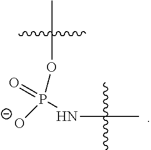 [SMILES: *NP(=O)([O-])O*]

As used herein, a thiophosphoramidate is used as understood by those skilled in the art and refers to a phosphate wherein one oxygen is replaced with a sulfur and one oxygen is replaced with an amide. The thiophosphoramidate can have the structure

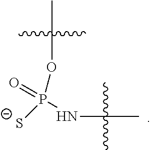 [SMILES: *NP(=O)([S-])O*]

As used herein, a phosphonoacetate is used as understood by those skilled in the art and refers to a phosphate wherein one oxygen is replaced with a —CH₂—C(═O)O⁻ or
CH₂—C(═O)OH. The phosphonoacetate can have the structure

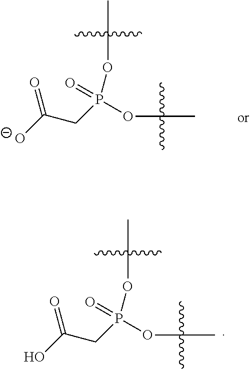 [SMILES: *OP(=O)(CC(=O)[O-])O* ; *OP(=O)(CC(=O)O)O*]

As used herein, a boranophosphate is used as understood by those skilled in the art and refers to a phosphate wherein one oxygen is replaced with a boron group. The boranophosphate can have the structure

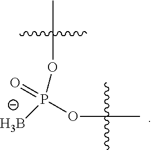

In some embodiments, the nucleosides are linked with all phosphodiester linkages. In some embodiments, the nucleosides are linked with all phosphorothioate linkages. In some embodiments, the nucleosides are linked with all methylphosphonate linkages. In some embodiments, the nucleosides are linked with all phosphoramidate linkages. In some embodiments, the nucleosides are linked with all thiophosphoramidate linkages. In some embodiments, the nucleosides are linked with all phosphonoacetate linkages. In some embodiments, the nucleosides are linked with all boranophosphate linkages. In some embodiments, the nucleosides are linked with a combination of phosphodiester and phosphorothioate linkages. In some embodiments, the nucleosides are linked with a combination of phosphodiester, phosphorothioate, methylphosphonate, phosphoramidate, thiophosphoramidate, phosphonoacetate, and boranophosphate linkages, including combinations where at least one type of linkage is not present.

Those skilled in the art understand that when the linkage is a non-phosphodiester linkage, the phosphorus can be a chiral center. For example, in a phosphorothioate, the phosphorus can be a (R)-stereocenter or a (S)-stereocenter. In some embodiments, each phosphorus of a non-phosphodiester linkage can be a (R)-stereocenter. In other embodiments, each phosphorus of a non-phosphodiester linkage can be a (S)-stereocenter. For example, in an oligonucleotide that has a phosphorothioate between each nucleoside, each phosphorothioate can be in the (S)-configuration. In still other embodiments, the oligonucleotide can include at least one non-phosphodiester linkage, wherein the phosphorus can be a (S)-stereocenter, and at least one non-phosphodiester linkage, wherein the phosphorus can be a (R)-stereocenter. In some embodiments, a particular linkage within an oligonucleotide may be present in a racemic mixture. In some embodiments, a particular linkage within an oligonucleotide may be present in an unequal mixture of (R) and (S) stereoisomers. For example, a particular linkage may be present where the ratio between (R) and (S) stereoisomers is 0%:100%, 10%:90%, 20%:80%, 30%:70%, 40%:60%, 50%:50%, 60%:40%, 70%:30%, 80%:20%, 90%:10%, 100%:0%, or any ratio in the range defined between any two aforementioned ratios. In some embodiments, a particular linkage within an oligonucleotide is enantiomerically pure, (R) enantiomerically pure, or (S) enantiomerically pure.

It is understood that, in any compound described herein having one or more chiral centers, if an absolute stereochemistry is not expressly indicated, then each center may independently be of (R)-configuration or (S)-configuration or a mixture thereof. Thus, the compounds provided herein may be enantiomerically pure, enantiomerically enriched, racemic mixture, diastereomerically pure, diastereomerically enriched, or a stereoisomeric mixture. Likewise, it is understood that, in any compound described, all tautomeric forms are also intended to be included.

As used herein, the term “antisense oligonucleotide” or “ASO” refers to a short oligonucleotide or nucleic acid polymer that targets single stranded messenger RNA (mRNA) transcripts by complementary base pairing. This binding activity of ASOs to a target mRNA can be used to prevent or modify translation of the mRNA, such as inhibiting 5′-G capping, manipulation or blocking of pre-mRNA splicing, or obstructing ribosomal binding. Another process by which ASOs downregulate gene expression is by forming DNA-RNA heteroduplexes (where the ASO comprises deoxyribose nucleotides), resulting in cleavage of the RNA strand by endogenous RNase H enzymes. An ASO may be about 12 to about 30 base pairs in length, such as 12, 13, 14, 15, 16, 17, 18, 19, 20, 21, 22, 23, 24, 25, 26, 27, 28, 29, or 30 base pairs in length. A common modification to ASOs is the substitution of the phosphodiester bonds to other linkages such as phosphorothioate bonds. This substitution limits degradation of the ASO by nucleases in the target cell, substantially improving half-life. Other modifications, such as those provided in this disclosure, to the linkages, sugar, or nucleobases are also employed to improve characteristics such as half-life, stability, safety, efficacy, solubility, melting temperature, selectivity, or permeability into a cell or across the blood-brain barrier.

The ASOs can further be modified with at least one additional moiety, such as a targeting moiety. In some embodiments, the targeting moiety is a lipophilic moiety. In some embodiments, the targeting moiety is a long chain fatty acid having a general structure of CH₃(CH₂)ₙ(CH)ₘCOOH, wherein n is a whole number ranging from 1 to 30, and wherein m is a whole number ranging from 1 to 30. Examples of a targeting moiety include, but are not limited to N-acetylgalactosamine (GalNAc, including, for example, a triantennary-GalNAc, including, for example, GalNAc3, GalNAc4, GalNAc5, GalNAc6 and/or GalNAc7), folic acid, cholesterol, tocopherol, vitamin E, or palmitate. Additional examples of long chain fatty acids include, but are not limited to, docohexanoic acid, docosanoic acid, linoleic acid (omega-6), linolenic acid (omega-3), oleic acid, octanoic acid, decanoyl acid, dodecanoyl acid, stearic acid, eicosanoic acid, and arachidonic acid. In some embodiments, the targeting moiety results in preferential targeting of the ASO to a certain organ or tissue, such as the liver, heart, lung, brain, bone, muscle, kidney, stomach, small intestine, large intestine, or pancreas. In some embodiments, a targeting moiety is conjugated to the 5′ end of the ASO. In some embodiments, a targeting moiety is conjugated to the 5′ phosphate of the ASO. In some embodiments, a targeting moiety is conjugated to the 3′ end of the ASO. In some embodiments, a targeting moiety is conjugated to the 3′ sugar hydroxyl of the ASO. In some embodiments, a targeting moiety is conjugated to the 5′ end and another targeting moiety is conjugated to the 3′ end of the ASO. In some embodiments, a second targeting moiety can be conjugated to a first targeting moiety. In some embodiments, a targeting moiety is attached with a linker. In some embodiments, the linker is a nucleotide, such as adenine, guanine, cytosine, thymine, or uracil nucleotides, or non-nucleoside linkers, including triethylene glycol (TEG), hexaethylene glycol (HEG), or alkyl amino linker.

GalNAc as used herein has the following structure

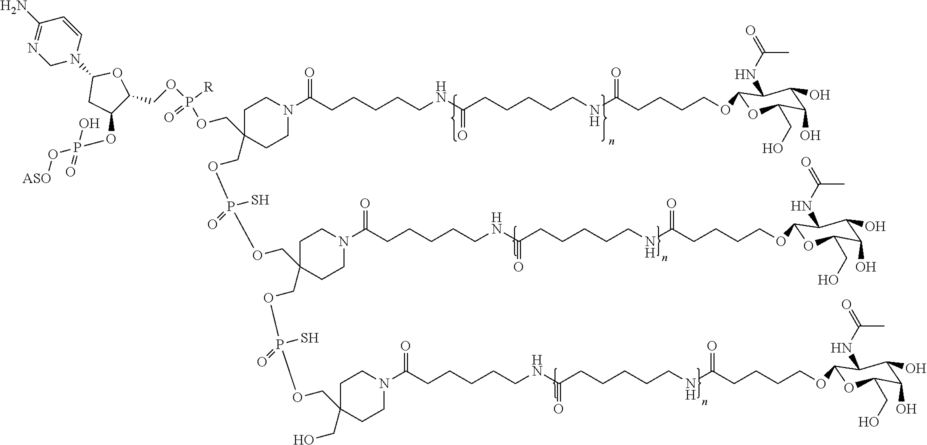

wherein R is OH or SH, and wherein n is any integer. In some embodiments, the deoxycytosine nucleotide shown in this structure linking the ASO to the GalNAc moiety is optional, and can be omitted. In some embodiments, n ranges from 0 to 10, such as 0, 1, 2, 3, 4, 5, 6, 7, 8, 9, or 10. For example, for GalNAc4, n=1; and for GalNAc6, n=2. However, it is to be understood that n may equal any integer and may be selected based on the desired characteristic of the targeting moiety.

As used herein, the term “gapmer” refers to an ASO that comprises both deoxyribose (DNA) and ribose (RNA)-based nucleotides wherein the DNA nucleotides are grouped together in the middle of the ASO sequence and flanked by the RNA nucleotides on both 5′ and 3′ ends. The DNA nucleotides are involved in RNase H-mediated cleavage of the target mRNA. Each of the flanking RNA nucleotides can be unmodified nucleotides or modified nucleotides to enhance ASO properties such as stability, resistance to nuclease degradation, base pairing efficiency, solubility, conformation/flexibility of the oligonucleotide, or melting temperature. The DNA nucleotides can also be modified nucleotides if the modifications do not affect base pairing to the target mRNA or RNase H activity to the desired threshold. The gapmer can also comprise base pair mismatches to the target sequence. In some embodiments, the base pair mismatches are in the outer RNA nucleotides, while the center DNA or RNA nucleotides of the gapmer are conserved.

As used herein, the term “mixmer” refers to an ASO that comprises both unmodified and modified DNA or RNA nucleotides wherein the unmodified nucleotides and modified nucleotides are distributed throughout the ASO sequence. In some embodiments, the unmodified nucleotides and modified nucleotides are alternating in the ASO sequence. In some embodiments, two, three, four, five, or six unmodified nucleotides, or two, three, four, five, or six modified nucleotides are grouped together in a sequence that otherwise contains alternating unmodified and modified nucleotides. Groups of contiguous unmodified nucleotides or contiguous modified nucleotides can be spaced across the ASO at regular intervals.

As used herein, the term “blockmer” refers to an ASO that comprises both unmodified and modified DNA or RNA nucleotides wherein the unmodified nucleotides are grouped together, and the modified nucleotides are grouped together. In some embodiments, the blockmer can comprise 1, 2, 3, 4, 5, 6, 7, or 8 unmodified nucleotides on the 5′ end of the ASO. In some embodiments, the blockmer can comprise 1, 2, 3, 4, 5, 6, 7, or 8 modified nucleotides on the 5′ end of the ASO. In some embodiments, the blockmer can comprise 1, 2, 3, 4, 5, 6, 7, or 8 unmodified nucleotides on the 3′ end of the ASO. In some embodiments, the blockmer can comprise 1, 2, 3, 4, 5, 6, 7, or 8 modified nucleotides on the 3′ end of the ASO.

As used herein, the term “Xmer” refers to an oligonucleotide or nucleic acid polymer that is “X” nucleotides long. For example, a 14mer is an oligonucleotide or nucleic acid polymer that is 14 nucleotides long, and a 20mer is an oligonucleotide or nucleic acid polymer that is 20 nucleotides long. In some embodiments, the “X” refers to the total number of nucleotides. In other embodiments, the “X” refers to the number of nucleotides involved in binding to the target, while the oligonucleotide or nucleic acid polymer may have additional nucleotides or components that are not involved in binding to the target.

In some embodiments, at least one ASO is used to treat liver disease. In some embodiments, the liver disease includes but is not limited to liver cancer, hepatocellular carcinoma (HCC), cholangiocarcinoma, hepatitis, hepatitis A, hepatitis B, hepatitis C, hepatitis D, or any combination thereof. In some embodiments, the at least one ASO is used to silence expression of a gene involved in a liver disease. In some embodiments, the gene is CD274. In some embodiments, the at least one ASO results in at least 0%, 10%, 20%, 30%, 40%, 50%, 60%, 70%, 80%, 90%, 91%, 92%, 93%, 94%, 95%, 96%, 97%, 98%, 99%, or 100% reduction in the disease or symptoms thereof.

The term “isolated” as used herein refers to material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

As used herein, the abbreviations for any protective groups and other compounds are used, unless indicated otherwise, in accord with their common usage.

It is to be understood that where compounds disclosed herein have unfilled valencies, then the valencies are to be filled with hydrogen or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

### Oligonucleotide Synthesis

Each of 2′-OMe, 2′-MOE, and LNA phosphoramidite monomers were procured from commercially-available sources. All the monomers were dried in vacuum desiccator with desiccants (P₂O₅, RT 24h). Universal solid supports (CPG) attached were obtained from ChemGenes. The chemicals and solvents for synthesis workflow were purchased from VWR/Sigma commercially-available sources and used without any purification or treatment. Solvent (Acetonitrile) and solutions (amidite and activator) were stored over molecular sieves during synthesis.

The control and target oligonucleotide sequences were synthesized on an Expedite 8909 synthesizer using the standard cycle written by the manufacturer with modifications as needed to wait steps and coupling steps. The solid support was controlled pore glass and the monomers contained standard protecting groups. Each chimeric oligonucleotide was individually synthesized using commercially available 5′-O-(4,4′-dimethoxytrityl)-3′-O-(2-cyanoethyl-N, N-diisopropyl) DNA, 2′-OMe, 2′-MOE and or LNA phosphoramidite monomers of 6-N-benzoyladenosine (A^{Bz}), 4-N-acetylcytidine (C^{Ac}), 2-N-isobutyrylguanosine (G^{iBu}), and Uridine (U) or Thymidine (T), according to standard solid phase phosphoramidite synthesis protocols. The 2′-O-Me-2,6, diaminopurine phosphoramidite was purchased from Glen Research. The phosphoramidites were prepared as 0.1 M solutions in anhydrous acetonitrile. 5-Ethylthiotetrazole was used as activator, 3% Dichloroacetic acid in dichloromethane was used to detritylate, acetic anhydride in THF and 16% N-methylimidazole in THF were used to cap, and DDTT ((dimethylamino-methylidene) amino)-3H-1,2,4-dithiazaoline-3-thione was used as the sulfur-transfer agent for the synthesis of oligoribonucleotide phosphorothioates. An extended coupling of 0.1M solution of phosphoramidite in CH₃CN in the presence of 5-(ethylthio)-1H-tetrazole activator to a solid bound oligonucleotide followed by extended capping, oxidation and deprotection to afford the modified oligonucleotides. The stepwise coupling efficiency of all modified phosphoramidites was more than 98.5%.

Deprotection and cleavage from the solid support was achieved with mixture of ammonia methylamine (1:1, AMA) for 15 min at 65° C., when the universal linker was used, the deprotection was left for 90 min at 65° C. or solid supports were heated with aqueous ammonia (28%) solution at 55° C. for 8 h to deprotect the base labile protecting groups. After filtering to remove the solid support, the deprotection solution was removed under vacuum in a GeneVac centrifugal evaporator. Tables 1-3 depicts exemplary structures of 2′-OMe, 2′-MOE, and LNA phosphoramidite monomers

TABLE 1
2′-OMe Phosphoramidite Monomers

| | |
|---|---|
| 2′-OMe-A phosphoramidite | 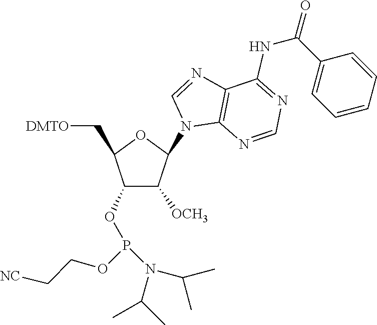 |
| 2′-OMe-C phosphoramidite | 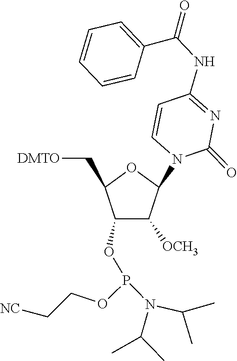 |
| 2′-OMe-G Phosphoramidite | 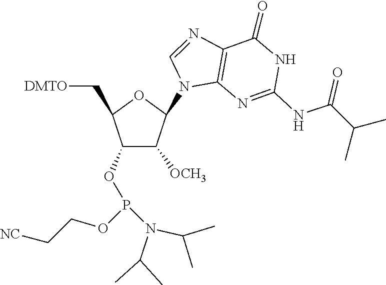 |
| 2′-OMe-U Phosphoramidite | 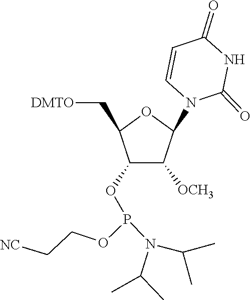 |

TABLE 2
2′-MOE Phosphoramidite Monomers

| | |
|---|---|
| 2′-MOE-A phosphoramidite | 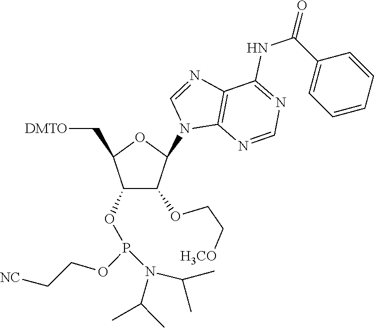 |
| 2′-MOE-(5m)C phosphoramidite | 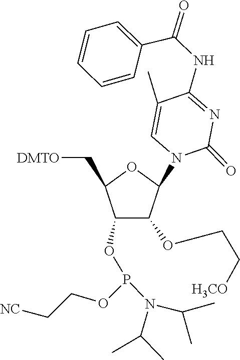 |
| 2′-MOE-G Phosphoramidite | 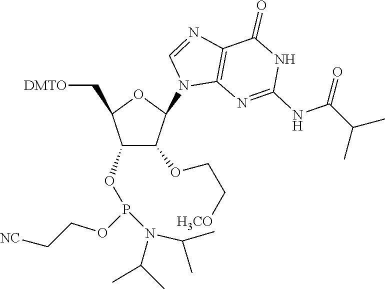 |
| 2′-MOE-T Phosphoramidite | 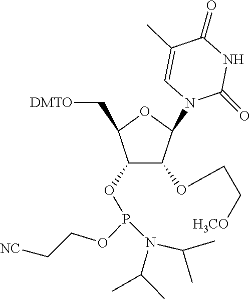 |

TABLE 3
2′-LNA Phosphoramidite Monomers

| | |
|---|---|
| LNA-A phosphoramidite | 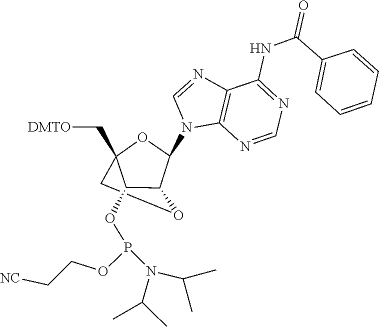 |
| LNA-(5m)C phosphoramidite | 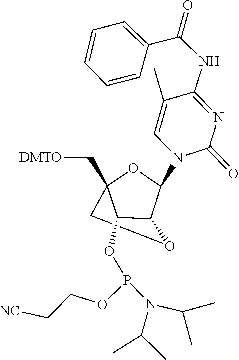 |
| LNA-G Phosphoramidite | 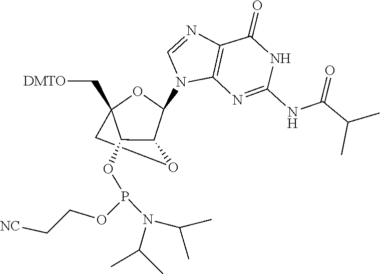 |
| LNA-T Phosphoramidite | 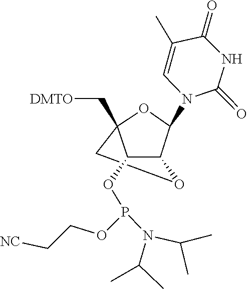 |

The AmNA and Scp-BNA phosphoramidite monomers of 6-N-benzoyladenosine (A^{Bz}), 4-N-acetylcytidine (C^{Ac}), 2-N-isobutyrylguanosine (G^{iBu}), and Thymidine (T) received from LUXNA Technologies. All the monomers were dried in a vacuum desiccator with desiccants (P₂O₅, at room temperature for 24 hours). For the AmNA-PS-DNA-PS and scp-BNA-PS-DNA-PS modifications, the synthesis was carried out on a 1 scale in a 3′ to 5′ direction with the phosphoramidite monomers diluted to a concentration of 0.12 M in anhydrous CH₃CN in the presence of 0.3 M 5-(benzylthio)-1H-tetrazole activator (coupling time 16-20 min) to a solid bound oligonucleotide followed by modified capping, oxidation and deprotection to afford the modified oligonucleotides. The stepwise coupling efficiency of all modified phosphoramidites was more than 97%. The DDTT (dimethylamino-methylidene) amino)-3H-1, 2, 4-dithiazaoline-3-thione was used as the sulfur-transfer agent for the synthesis of the oligoribonucleotide phosphorothioates. Oligonucleotide-bearing solid supports were washed with 20% DEA solution in acetonitrile for 15 min then the column was washed thoroughly with AcCN. The support was heated at 65° C. with Diisopropylamine:water:Methanol (1:1:2) for 5 h in heat block to cleave from the support and deprotect the base labile protecting groups. Tables 4 and 5 depicts exemplary structures of the AmNA and Scp-BNA phosphoramidite monomers.

TABLE 4
am-NCH₃ Phosphoramidite Monomers

| | |
|---|---|
| am-NCH₃-A phosphoramidite | 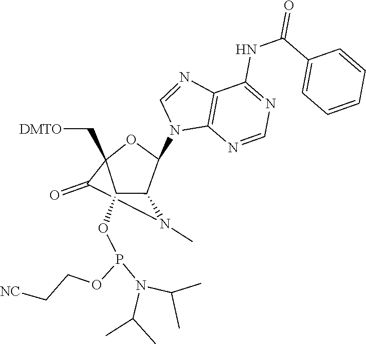 |
| am-NCH₃-(5m)C phosphoramidite | 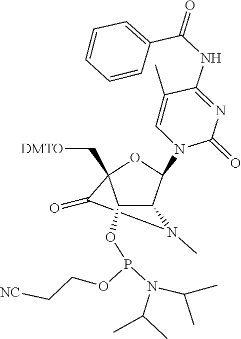 |
| am-NCH₃-G Phosphoramidite | 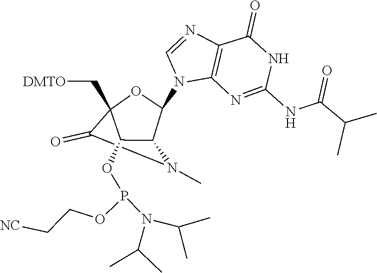 |
| am-NCH₃-T Phosphoramidite | 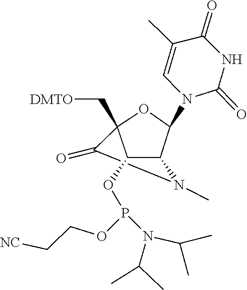 |

TABLE 5
Scp-BNA Phosphoramidite Monomers

| | |
|---|---|
| Scp-BNA-A phosphoramidite | 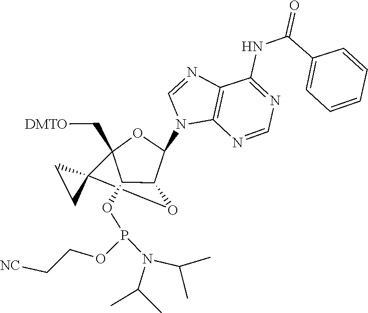 |
| Scp-BNA-(5m)C phosphoramidite | 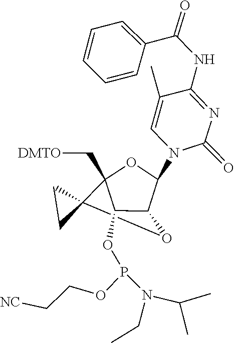 |
| Scp-BNA-G Phosphoramidite | 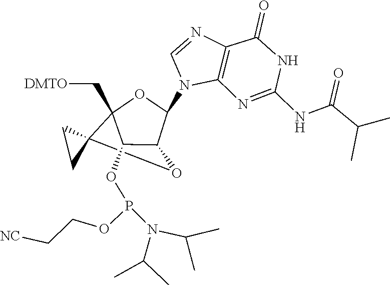 |
| Scp-BNA-T Phosphoramidite | 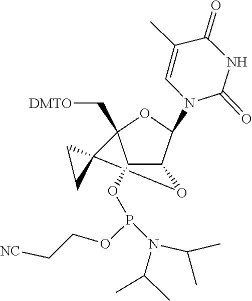 |

The cholesterol, tocopherol phosphoramidite, and solid supports were received from ChemGenes. The cholesterol and Tocopherol conjugated oligonucleotides were obtained by starting solid phase synthesis on cholesterol and Tocopherol supports attached on TEG linker for 3′-conjugation while final coupling of the phosphoramidite provided the 5′-conjugated oligonucleotides.

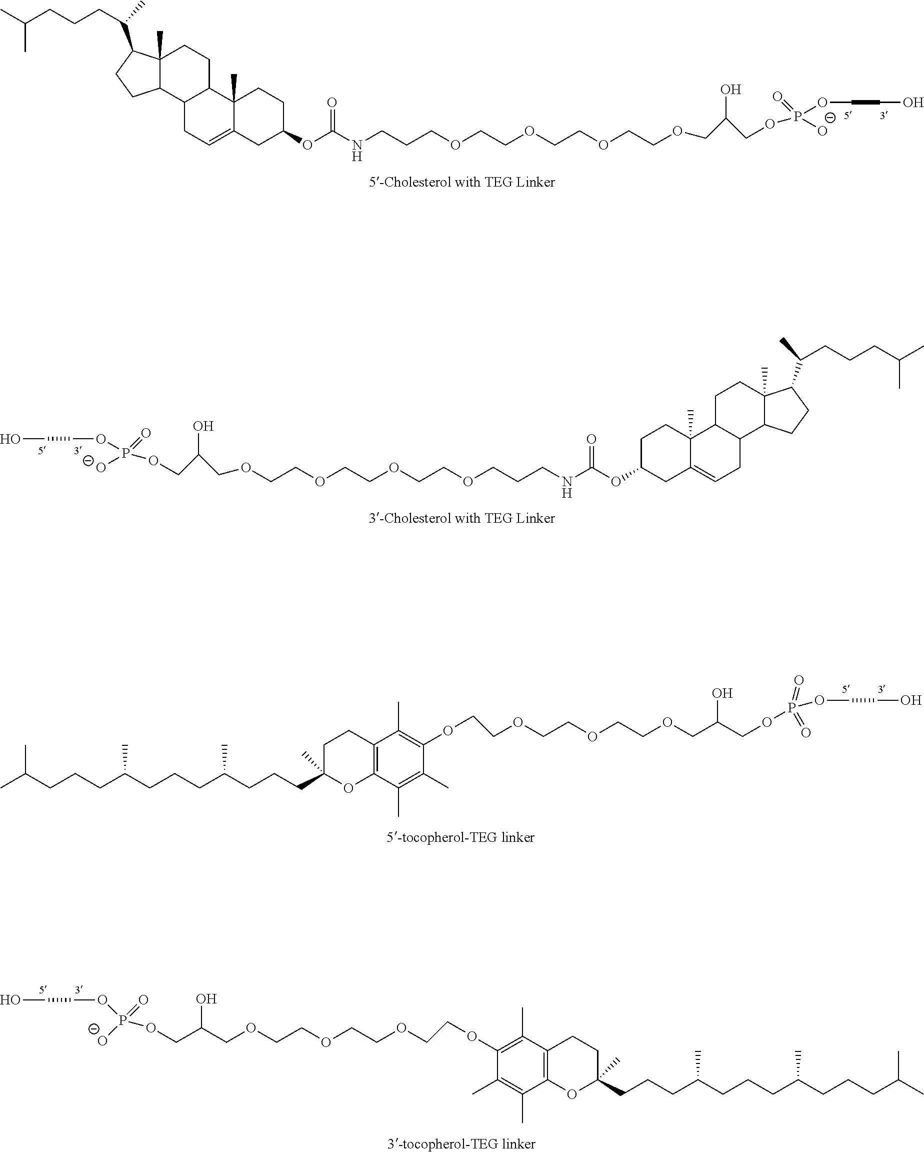

### Quantitation of Crude Oligomer or Raw Analysis

Samples were dissolved in deionized water (1.0 mL) and quantified as follows: Blanking was first performed with water alone (1.0 mL), then 20 μL of sample and 980 μL of water were mixed well in a microfuge tube, transferred to cuvette and absorbance reading obtained at 260 nm. The crude material was dried and stored at −20° C.

### Crude HPLC/LC-MS Analysis

The 0.1 OD of the crude samples were used for crude MS analysis. After confirming the crude LC-MS data, the purification step was performed.

### HPLC Purification

The Phosphodiester (PO), Phosphorothioate (PS) and chimeric modified oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.8 M NaBr, pH 8.5 (buffer B). Fractions containing full-length oligonucleotides were pooled, desalted and lyophilized.

The conjugated oligonucleotides were purified by an in-house packed RPC-Source15 reverse-phase column. The buffers were 20 mM sodium acetate in 10% CH₃CN, (buffer A) and CH₃CN (buffer B). Fractions containing full-length oligonucleotides were pooled, desalted and lyophilized.

### Desalting of Purified Oligomer

The purified dry oligomer was then desalted using Sephadex G-25 M (Amersham Biosciences). The cartridge was conditioned with 10 mL of deionized water thrice. The purified oligonucleotide dissolved thoroughly in 2.5 mL deionized water was applied to the cartridge with very slow drop wise elution. The salt free oligomer was eluted with 3.5 mL deionized water directly into a screw cap vial.

### Final HPLC and Electrospray LC/MS Analysis

Approximately 0.10 OD of oligomer is dissolved in water and then pipetted in special vials for IEX-HPLC and LC/MS analysis. Analytical HPLC and ES LC-MS established the integrity of the chimeric oligonucleotides.

The cholesterol and tocopherol conjugated sequences were analyzed by high-performance liquid chromatography (HPLC) on a Luna C8 reverse-phase column. The buffers were 20 mM NaOAc in 10% CH₃CN (buffer A) and 20 mM NaOAc in 70% CH₃CN (buffer B). Analytical HPLC and ES LC-MS established the integrity of the conjugated oligonucleotides

### Post Synthesis Conjugation:

5′-Folate conjugated ASOs: To a solution of 5′-hexylamino ASO in 0.1 M sodium tetraborate buffer, pH 8.5 (2 mM) a solution of Folate-NHS ester (3 mole equivalent) dissolved in DMSO (40 mM) was added, and the reaction mixture was stirred at room temperature for 3 h. The Reaction mixture concentrated under reduced pressure. The residue was dissolved in water and purified by HPLC on a strong anion exchange column (GE Healthcare Bioscience, Source 30Q, 30 μm, 2.54×8 cm, A=100 mM ammonium acetate in 30% aqueous CH₃CN, B=1.8 M NaBr in A, 0-60% of B in 60 min, flow 10 mL/min). The residue was desalted by in house packed Sephadex G-25 column to yield the 5′-Folate conjugated ASOs in an isolated yield of 62-80%. The folate conjugated ASOs were characterized by IEX-HPLC and Thermo Fischer ESI-LC-MS system. Table 6 depicts exemplary nucleic acids and structures.

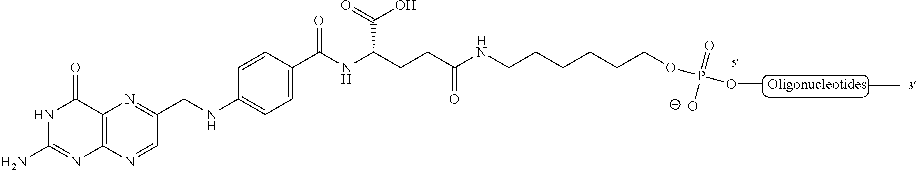

TABLE 6
Abbreviations for nucleic acid structures

| Abbre- | | |
|---|---|---|
| viation | Name | Structure |
| A | Adenine | 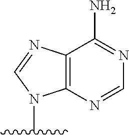 [SMILES: *n1cnc2c(N)ncnc21] |
| G | Guanine | 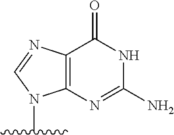 [SMILES: *n1cnc2c(=O)[nH]c(N)nc21] |
| C | Cytosine | 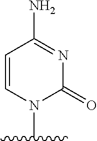 [SMILES: *n1ccc(N)nc1=O] |
| U | Uracil | 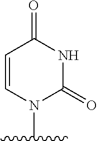 [SMILES: *n1ccc(=O)[nH]c1=O] |
| T | Thymine | 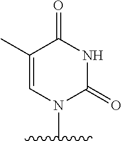 [SMILES: *n1cc(C)c(=O)[nH]c1=O] |
| (5m)C | 5-methyl- cytosine | 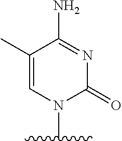 [SMILES: *n1cc(C)c(N)nc1=O] |
| DAP | 2,6- diamino- purine | 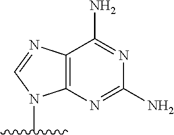 [SMILES: *n1cnc2c(N)nc(N)nc21] |
| d | Deoxy | 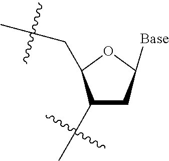 |
| ps | Phos- phorothioate | 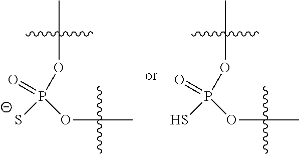 [SMILES: *OP(=O)([S-])O* ; *OP(=O)(S)O*] |
| ln | LNA | 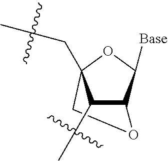 |
| am | AmNA | 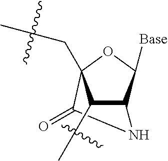 |
| scp | Scp-BNA | 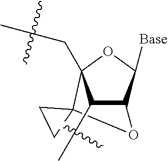 |
| m | 2’-OMe | 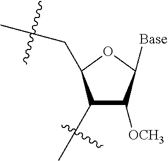 |
| moe | 2’-MOE | 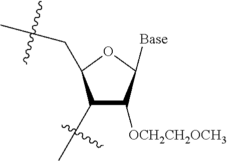 |
| cet | cEt | 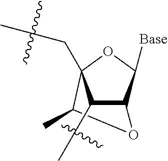 |

Any of the structures shown in Table 6 can be combined with any base, thereby generating various combinations of structures. For example, using the abbreviations and structures from Table 6, one skilled in the art understands that the abbreviation “AmG” represents

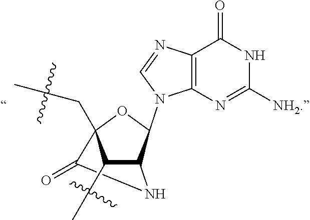

Furthermore, additional structures not depicted in the tables, but described elsewhere throughout the application may be used and combined with any base described in the tables or elsewhere throughout the application.

### Pharmaceutical Compositions

Some embodiments described herein relate to pharmaceutical compositions that comprise, consist essentially of, or consist of an effective amount of an oligonucleotide described herein and a pharmaceutically acceptable carrier, excipient, or combination thereof. A pharmaceutical composition described herein is suitable for human and/or veterinary applications.

The terms “function” and “functional” as used herein refer to a biological, enzymatic, or therapeutic function.

The terms “effective amount” or “effective dose” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “pharmaceutically acceptable salts” includes relatively non-toxic, inorganic and organic acid, or base addition salts of compositions, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

“Formulation”, “pharmaceutical composition”, and “composition” as used interchangeably herein are equivalent terms referring to a composition of matter for administration to a subject.

The term “pharmaceutically acceptable” means compatible with the treatment of a subject, and in particular, a human.

The terms “agent” refers to an active agent that has biological activity and may be used in a therapy. Also, an “agent” can be synonymous with “at least one agent,” “compound,” or “at least one compound,” and can refer to any form of the agent, such as a derivative, analog, salt or a prodrug thereof. The agent can be present in various forms, components of molecular complexes, and pharmaceutically acceptable salts (e.g., hydrochlorides, hydrobromides, sulfates, phosphates, nitrates, borates, acetates, maleates, tartrates, and salicylates). The term “agent” can also refer to any pharmaceutical molecules or compounds, therapeutic molecules or compounds, matrix forming molecules or compounds, polymers, synthetic molecules and compounds, natural molecules and compounds, and any combination thereof.

The term “subject” as used herein has its ordinary meaning as understood in light of the specification and refers to an animal that is the object of treatment, inhibition, or amelioration, observation or experiment. “Animal” has its ordinary meaning as understood in light of the specification and includes cold- and warm-blooded vertebrates and/or invertebrates such as fish, shellfish, or reptiles and, in particular, mammals. “Mammal” has its ordinary meaning as understood in light of the specification, and includes but is not limited to mice, rats, rabbits, guinea pigs, dogs, cats, sheep, goats, cows, horses, primates, such as humans, monkeys, chimpanzees, or apes. In some embodiments, the subject is human.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration. Pharmaceutical compositions can also be administered to isolated cells from a patient or individual, such as T cells, Natural Killer cells, B cells, macrophages, lymphocytes, stem cells, bone marrow cells, or hematopoietic stem cells.

The pharmaceutical compound can also be administered in a local rather than systemic manner, for example, via injection of the compound directly into an organ, tissue, cancer, tumor or infected area, often in a depot or sustained release formulation. Furthermore, one may administer the compound in a targeted drug delivery system, for example, in a liposome coated with a tissue specific antibody. The liposomes may be targeted to and taken up selectively by the organ, tissue, cancer, tumor, or infected area.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the liver.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “adjuvant” as used herein refers to a substance, compound, or material that stimulates the immune response and increase the efficacy of protective immunity and is administered in conjunction with an immunogenic antigen, epitope, or composition. Adjuvants serve to improve immune responses by enabling a continual release of antigen, up-regulation of cytokines and chemokines, cellular recruitment at the site of administration, increased antigen uptake and presentation in antigen presenting cells, or activation of antigen presenting cells and inflammasomes. Commonly used adjuvants include but are not limited to alum, aluminum salts, aluminum sulfate, aluminum hydroxide, aluminum phosphate, calcium phosphate hydroxide, potassium aluminum sulfate, oils, mineral oil, paraffin oil, oil-in-water emulsions, detergents, MF59®, squalene, AS03, α-tocopherol, polysorbate 80, AS04, monophosphoryl lipid A, virosomes, nucleic acids, polyinosinic:polycytidylic acid, saponins, QS-21, proteins, flagellin, cytokines, chemokines, IL-1, IL-2, IL-12, IL-15, IL-21, imidazoquinolines, CpG oligonucleotides, lipids, phospholipids, dioleoyl phosphatidylcholine (DOPC), trehalose dimycolate, peptidoglycans, bacterial extracts, lipopolysaccharides, or Freund's Adjuvant, or any combination thereof.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

### Methods of Use

Some embodiments disclosed herein related to selecting a subject or patient in need. In some embodiments, a patient is selected who is in need of treatment, inhibition, amelioration, prevention or slowing of diseases or conditions associated with PD-L1 dysregulation. In some embodiments, such diseases or conditions associated with PD-L1 dysregulation may include, for example, cancer, HCC, viral infections, or HBV. In some embodiments, a patient is selected who has previously been treated for the disease or disorder described herein. In some embodiments, a patient is selected who has previously been treated for being at risk for the disease or disorder described herein. In some embodiments, a patient is selected who has developed a recurrence of the disease or disorder described herein. In some embodiments, a patient is selected who has developed resistance to therapies for the disease or disorder described herein. In some embodiments, a patient is selected who may have any combination of the aforementioned selection criteria.

Compounds disclosed herein can be evaluated for efficacy and toxicity using known methods. A non-limiting list of potential advantages of an oligonucleotide described herein include improved stability, increased safety profile, increased efficacy, increased binding to the target, increased specificity for the target (for example, a cancer cell or virally infected cell).

The terms “treating,” “treatment,” “therapeutic,” or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

Some embodiments described herein relate to a method of treating, inhibiting, ameliorating, preventing, or slowing the disease or disorder described herein. In some embodiments, the methods include administering to a subject identified as suffering from the disease or disorder described herein an effective amount of an ASO described herein, or a pharmaceutical composition that includes an effective amount of an ASO as described herein. Other embodiments described herein relate to using an ASO as described herein in the manufacture of a medicament for treating, inhibiting ameliorating, preventing, or slowing the disease or disorder described herein. Still other embodiments described herein relate to the use of an ASO as described herein or a pharmaceutical composition that includes an effective amount of an ASO as described herein for treating, inhibiting ameliorating, preventing, or slowing the disease or disorder described herein.

Some embodiments described herein relate to a method for inhibiting replication of a cancer cell or a virus that can include contacting the cell or virus or administering to a subject identified as suffering from a cancer or a viral infection with an effective amount of an ASO described herein, or a pharmaceutical composition that includes an effective amount of an ASO described herein. Other embodiments described herein relate to the use of an effective amount of an ASO described herein, or a pharmaceutical composition that includes an effective amount of an ASO described herein in the manufacture of a medicament for inhibiting replication of a cancer cell or virus. Still other embodiments described herein relate to an effective amount of an ASO described herein, or a pharmaceutical composition that includes an effective amount of an ASO described herein for inhibiting replication of a cancer cell or virus. In some embodiments, the cancer cell is an HCC cell. In some embodiments, the virus is hepatitis B.

Some embodiments described herein relate to a method for inhibiting cell proliferation, such as inhibiting cell proliferation of a cancer cell or cell infected with a virus, that can include administering to a subject identified as suffering from a disease wherein inhibiting cell proliferation is desirable with an effective amount of an ASO described herein, or a pharmaceutical composition that includes effective amount of an ASO described herein. Other embodiments described herein relate to the use of an effective amount of an oligonucleotide described herein, or a pharmaceutical composition that includes an effective amount of an ASO described herein in the manufacture of a medicament for inhibiting cell proliferation, such as inhibiting cell proliferation of a cancer cell or cell infected with a virus. Still other embodiments described herein relate to an effective amount of an ASO described herein, or a pharmaceutical composition that includes an effective amount of an ASO described herein for inhibiting cell proliferation, such as inhibiting cell proliferation of a cancer cell or cell infected with a virus. In some embodiments, the cancer cell is an HCC cell. In some embodiments, the cell infected with a virus is infected with hepatitis B virus.

Some embodiments described herein relate to a method of inducing apoptosis of a cell (for example, a cancer cell or cell infected with a virus) that can include contacting the cell with an effective amount of an ASO described herein, or a pharmaceutical composition that includes an effective amount of an ASO as described herein. Other embodiments described herein relate to using an effective amount of an ASO as described herein or a pharmaceutical composition that includes an effective amount of an ASO in the manufacture of a medicament for inducing apoptosis of a cell, such as a cancer cell or cell infected with a virus. Still other embodiments described herein relate to the use of an effective amount of an ASO as described herein or a pharmaceutical composition that includes an effective amount of an ASO as described herein for inducing apoptosis of a cell, such as a cancer cell or cell infected with a virus. In some embodiments, the cancer cell is an HCC cell. In some embodiments, the cell infected with a virus is infected with hepatitis B virus.

Some embodiments described herein relate to a method of decreasing the viability of a cell (for example, a cancer cell or cell infected with a virus) that can include contacting the cell with an effective amount of an ASO described herein, or a pharmaceutical composition that includes an effective amount of an ASO as described herein. Other embodiments described herein relate to using an ASO as described herein in the manufacture of a medicament for decreasing the viability of a cell, such as a cancer cell or cell infected with a virus. Still other embodiments described herein relate to the use of an effective amount of an ASO as described herein or a pharmaceutical composition that includes an effective amount of an ASO as described herein for decreasing the viability of a cell, such as a cancer cell or cell infected with a virus. In some embodiments, the cancer cell is an HCC cell. In some embodiments, the cell infected with a virus is infected with hepatitis B virus.

In some embodiments, the effective amount of an ASO for a human subject is 1, 10, 50, 100, 200, 300, 400, 500, 600, 700, 800, 900, 1000 or 1, 10, 20, 30, 40, 50, 60, 70, 80, 90, 100, 110, 120, 130, 140, 150, 160, 170, 180, 190, 200, 300, 400, 500, 600, 700, 800, 900, 1000 mg or any amount within the range defined by any two aforementioned amounts. In some embodiments, the effective amount of an ASO for a human subject is 1, 10, 50, 100, 200, 300, 400, 500, 600, 700, 800, 900, 1000 ng/kg, or 1, 10, 20, 30, 40, 50, 60, 70, 80, 90, 100, 110, 120, 130, 140, 150, 160, 170, 180, 190, 200, 300, 400, 500, 600, 700, 800, 900, 1000 μg/kg or any amount within the range defined by any two aforementioned amounts. In some embodiments, the effective amount of an ASO is dosed more than one time. In some embodiments, the ASO dose is administered every 1, 2, 3, 4, 5, 6, 7 days, or 1, 2, 3, 4 weeks, or 1, 2, 3, 4, 5, 6, 7, 8, 9, 10, 11, 12 months, or 1, 2, 3, 4, 5 years, or any period or combination thereof within the range defined by any two aforementioned times. In some embodiments, at least one loading dose and at least one maintenance dose is administered to the subject, where the at least one loading dose is a higher dose of the ASO than the at least one maintenance dose.

As used herein, the term “combination therapy” is intended to define therapies which comprise the use of a combination of two or more pharmaceutical compounds/agents or therapies. Thus, references to “combination therapy”, “combinations” and the use of compounds/agents “in combination” in this application may refer to compounds/agents that are administered as part of the same overall treatment regimen. As such, the dosage or timing of each of the two or more compounds/agents may differ: each may be administered at the same time or at different times. Accordingly, the compounds/agents of the combination may be administered sequentially (e.g. before or after) or simultaneously, either in the same pharmaceutical formulation (i.e. together), or in different pharmaceutical formulations (i.e. separately). Each of the two or more compounds/agents in a combination therapy may also differ with respect to the route of administration.

The term “inhibitor”, as used herein, refers to an enzyme inhibitor or receptor inhibitor which is a molecule that binds to an enzyme or receptor, and decreases and/or blocks its activity. The term may relate to a reversible or an irreversible inhibitor.

Cancer may be treated with surgery, radiation therapy, chemotherapy, targeted therapies, immunotherapy or hormonal therapies. Any of these mentioned therapies may be used in conjunction with another therapy as a combination therapy. Chemotherapeutic compounds include but are not limited to alemtuzumab, altretamine, azacitidine, bendamustine, bleomycin, bortezomib, busulfan, cabazitaxel, capecitabine, carboplatin, carmofur, carmustine, chlorambucil, chlormethine, cisplatin, cladribine, clofarabine, cyclophosphamide, cytarabine, dacarbazine, dactinomycin, daunorubicin, decitabine, denosumab, docetaxel, doxorubicin, epirubicin, estramustine, etoposide, everolimus, floxuridine, fludarabine, fluorouracil, fotemustine, gemcitabine, gemtuzumab, hydroxycarbamide, ibritumomab, idarubicin, ifosfamide, irinotecan, ixabepilone, lomustine, melphalan, mercaptopurine, methotrexate, mitomycin, mitoxantrone, nedaplatin, nelarabine, ofatumumab, oxaliplatin, paclitaxel, pemetrexed, pentostatin, pertuzumab, procarbazine, raltitrexed, streptozotocin, tegafur, temozolomide, temsirolimus, teniposide, tioguanine, topotecan, tositumomab, valrubicin, vinblastine, vincristine, vindesine, vinflunine, or vinorelbine, or any combination thereof.

As used herein, the term “protein kinase inhibitor” refers to inhibitors of protein kinases, serine/threonine kinases, tyrosine kinases, or dual-specificity kinases for the treatment of cancer or other illness. In some embodiments, the protein kinase inhibitor is a small molecule, compound, polysaccharide, lipid, peptide, polypeptide, protein, antibody, nucleoside, nucleoside analog, nucleotide, nucleotide analog, nucleic acid, or oligonucleotide. In some embodiments, the protein kinase inhibitor includes but is not limited to acalabrutinib, adavosertib, afatinib, alectinib, axitinib, binimetinib, bosutinib, brigatinib, cediranib, ceritinib, cetuximab, cobimetinib, crizotinib, cabozantinib, dacomitinib, dasatinib, entrectinib, erdafitinib, erlotinib, fostamatinib, gefitinib, ibrutinib, imatinib, lapatinib, lenvatinib, lestaurtinib, lortatinib, masitinib, momelotinib, mubritinib, neratinib, nilotinib, nintedanib, olmutinib, osimertinib, pacritinib, panitumumab, pazopanib, pegaptanib, ponatinib, radotinib, regorafenib, rociletinib, ruxolitinib, selumetinib, semaxanib, sorafenib, sunitinib, SU6656, tivozanib, toceranib, trametinib, trastuzumab, vandetanib, or vemurafenib, or any combination thereof.

As used herein, the term “checkpoint inhibitor” refers to an immunotherapy that targets immune checkpoints to stimulate immune function. In some embodiments, the checkpoint inhibitor is a small molecule, compound, polysaccharide, lipid, peptide, polypeptide, protein, antibody, nucleoside, nucleoside analog, nucleotide, nucleotide analog, nucleic acid, or oligonucleotide. In some embodiments, the immune checkpoint is the PD-1/PD-L1 checkpoint. In some embodiments, the PD-1 checkpoint includes but is not limited to nivolumab, pembrolizumab, spartalizumab, cemiplimab, camrelizumab, sintilimab, tislelizumab, toripalimab, AMP-224 or AMP-514, or any combination thereof. In some embodiments, the PD-L1 checkpoint inhibitor includes but is not limited to atezolizumab, avelumab, durvalumab, KN035, AUNP12, CA-170, or BMS-986189, or any combination thereof. In some embodiments, the immune checkpoint is the CTLA-4 checkpoint. In some embodiments, the CTLA-4 checkpoint inhibitor includes but is not limited to ipilimumab or tremilimumab, or any combination thereof.

As used herein, the term “VEGF inhibitor” refers to inhibitors of vascular endothelial growth factor (VEGF) or a VEGF receptor (VEGFR). In some embodiments, the VEGF inhibitor is a small molecule, compound, polysaccharide, lipid, peptide, polypeptide, protein, antibody, nucleoside, nucleoside analog, nucleotide, nucleotide analog, nucleic acid, or oligonucleotide. In some embodiments, the VEGF inhibitor includes but is not limited to aflibercept, axitinib, bevacizumab, brivanib, cabozantinib, cediranib, lenvatinib, linifinib, nintedanib, pazopanib, ponatinib, ramucirumab, regorafenib, semaxanib, sorafenib, sunitinib, tivozanib, toceranib, or vandetanib, or any combination thereof.

As used herein, the term “antiviral medication” refers to a pharmaceutical composition administered to treat a viral infection. In some embodiments, the viral infection is caused by adenovirus, Ebola virus, coronavirus, Epstein-Barr virus (EBV), Friend virus, hantavirus, hepatitis B virus (HBV), hepatitis C virus (HCV), herpes simplex virus, human immunodeficiency virus (HIV), human metapneumovirus, human papillomavirus (HPV), influenza virus, Japanese encephalitis virus, Kaposi's sarcoma-associated herpesvirus, lymphocytic choriomeningitis virus, parainfluenza virus, rabies virus, respiratory syncytial virus, rhinovirus, varicella zoster virus. In some embodiments, the antiviral medication is a small molecule, compound, polysaccharide, lipid, peptide, polypeptide, protein, antibody, nucleoside, nucleoside analog, nucleotide, nucleotide analog, nucleic acid, or oligonucleotide. In some embodiments, the antiviral medication is an interferon, a capsid assembly modulator, a sequence specific oligonucleotide, an entry inhibitor, or a small molecule immunomodulatory. In some embodiments, the antiviral medication includes but is not limited to AB-423, AB-506, ABI-H2158, ABI-H0731, acyclovir, adapromine, adefovir, alafenamide, amantadine, asunaprevir, baloxavir marboxil, beclabuvir, boceprevir, brivudine, cidofovir, ciluprevir, clevudine, cytarabine, daclatasvir, danoprevir, dasabuvir, deleobuvir, dipivoxil, edoxudine, elbasvir, entecavir, faldaprevir, famciclovir, favipiravir, filibuvir, fomivirsen, foscarnet, galidesivir, ganciclovir, glecaprevir, GLS4, grazoprevir, idoxuridine, imiquimod, IFN-α, interferon alfa 2b, JNJ-440, JNJ-6379, lamivudine, laninamivir, ledipasvir, mericitabine, methisazone, MK-608, moroxydine, narlaprevir, NITD008, NZ-4, odalasvir, ombitasvir, oseltamivir, paritaprevir, peginterferon alfa-2a, penciclovir, peramivir, pibrentasvir, pimodivir, pleconaril, podophyllotoxin, presatovir, radalbuvir, ravidasvir, remdesivir, REP 2139, REP 2165, resiquimod, RG7907, ribavirin, rifampicin, rimantadine, ruzasvir, samatasvir, setrobuvir, simeprevir, sofosbuvir, sorivudine, sovaprevir, taribavirin, telaprevir, telbivudine, tenofovir, tenofovir disoproxil, triazavirin, trifluridine, tromantadine, umifenovir, uprifosbuvir, valaciclovir, valgancicovir, vaniprevir, vedroprevir, velpatasvir, vidarabine, voxilaprevir, or zanamivir, or any combination thereof.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

The invention is generally disclosed herein using affirmative language to describe the numerous embodiments. The invention also includes embodiments in which subject matter is excluded, in full or in part, such as substances or materials, method steps and conditions, protocols, or procedures.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1: Antisense Oligonucleotide (ASO) Design

ASOs were selected having 14-20 nucleotides in length. To account for optimal gapmer design, mismatches were allowed only in the outer flank regions of the ASO, as shown in Table 7. The typical DNA nucleotide central gap was 8, 9 or 10 nucleotides in length. Therefore, the central 10 or 11 nucleotides were kept fully conserved. Table 7 summarizes the allowed mismatch position for ASOs of different lengths. ASOs of 14 or 15 nucleotides in length were not allowed to have mismatches. ASOs of 16 or 17 nucleotides in length included 1 or fewer mismatches within the three outer RNA nucleotides on either 5′ or 3′ end. ASOs of 18 or 19 nucleotides in length included 2 or fewer mismatches within the four outer RNA nucleotides on either 5′ or 3′ end. ASOs of 20 nucleotides in length included 2 or fewer mismatches within the five outer RNA nucleotides on either 5′ or 3′ end.

TABLE 7
Allowed mismatch positions on ASOs

| ASO | Maximum | Fully conserved | Allowed mismatch |
|---|---|---|---|
| length | mismatches | positions | positions |
| 14 | 0 | 3-12 | None |
| 15 | 0 | 3-13 | None |
| 16 | 1 | 4-13 | 1, 2, 3, 14, 15, 16 |
| 17 | 1 | 4-14 | 1, 2, 3, 15, 16, 17 |
| 18 | 2 | 5-14 | 1, 2, 3, 4, 15, 16, 17, 18 |
| 19 | 2 | 5-15 | 1, 2, 3, 4, 16, 17, 18, 19 |
| 20 | 2 | 6-15 | 1, 2, 3, 4, 5, 16, 17, 18, 19, 20 |

### Example 2: ASOs Targeting the Human CD274 Gene (PD-L1)

ASOs were designed using the human CD274 mRNA transcript (NCBI accession number NM_014143.4, 3634 nt in length, SEQ ID NO: 1) as the template. 14-mers are depicted in Table 8 (SEQ ID NOs: 2-28). 15-mers are depicted in Table 9 (SEQ ID NOs: 29-46). 16-mers are depicted in Table 10 (SEQ ID NOs: 47-86 and 240-301). 17-mers are depicted in Table 11 (SEQ ID NOs: 87-114). 18-mers are depicted in Table 12 (SEQ ID NOs: 115-165). 19-mers are depicted in Table 13 (SEQ ID NOs: 166-204). 20-mers are depicted in Table 14 (SEQ ID NO: 205-239).

Any of the ASOs listed here, and the individual nucleobases, sugars, linkages, nucleosides, nucleotides and additional moieties thereof, can be constructed and used with any of the modifications described herein. The sequences listed in Tables 8-14 and SEQ ID NOs: 2-301 represent the unmodified oligonucleotide sequence prior to application of modifications.

TABLE 8
CD274 ASOs - 14-mers

| Target | Target | | SEQ |
|---|---|---|---|
| start | positions | Oligo Sequence | ID |
| position | spanned | (5′ → 3′) | NO: |
| 70 | 83-70 | GCAAATATCCTCAT | 2 |
| 71 | 84-71 | AGCAAATATCCTCA | 3 |
| 72 | 85-72 | CAGCAAATATCCTC | 4 |
| 1399 | 1412-1399 | CTCAAAATAAATAG | 5 |
| 1400 | 1413-1400 | ACTCAAAATAAATA | 6 |
| 1401 | 1414-1401 | GACTCAAAATAAAT | 7 |
| 1402 | 1415-1402 | AGACTCAAAATAAA | 8 |
| 1403 | 1416-1403 | CAGACTCAAAATAA | 9 |
| 1404 | 1417-1404 | ACAGACTCAAAATA | 10 |
| 1405 | 1418-1405 | CACAGACTCAAAAT | 11 |
| 2363 | 2376-2363 | CATTTAAATAGAAA | 12 |
| 2623 | 2636-2623 | AATAAATAAAAATA | 13 |
| 3528 | 3541-3528 | ATTAAATTAATGCA | 14 |
| 3529 | 3542-3529 | TATTAAATTAATGC | 15 |
| 3530 | 3543-3530 | TTATTAAATTAATG | 16 |
| 3531 | 3544-3531 | TTTATTAAATTAAT | 17 |
| 364 | 377-364 | TCTGTGATCTGAAG | 18 |
| 433 | 446-433 | ATTCGCTTGTAGTC | 19 |
| 452 | 465-452 | GGCATTGACTTTCA | 20 |
| 453 | 466-453 | GGGCATTGACTTTC | 21 |
| 454 | 467-454 | GGGGCATTGACTTT | 22 |
| 455 | 468-455 | TGGGGCATTGACTT | 23 |
| 456 | 469-456 | ATGGGGCATTGACT | 24 |
| 457 | 470-457 | TATGGGGCATTGAC | 25 |
| 458 | 471-458 | GTATGGGGCATTGA | 26 |
| 641 | 654-641 | GCTGGTCACATTGA | 27 |
| 642 | 655-642 | TGCTGGTCACATTG | 28 |

TABLE 9
CD274 ASOs - 15mers

| Target | Target | | SEQ |
|---|---|---|---|
| start | positions | Oligo Sequence | ID |
| position | spanned | (5′ → 3′) | NO: |
| 70 | 84-70 | AGCAAATATCCTCAT | 29 |
| 71 | 85-71 | CAGCAAATATCCTCA | 30 |
| 1399 | 1413-1399 | ACTCAAAATAAATAG | 31 |
| 1400 | 1414-1400 | GACTCAAAATAAATA | 32 |
| 1401 | 1415-1401 | AGACTCAAAATAAAT | 33 |
| 1402 | 1416-1402 | CAGACTCAAAATAAA | 34 |
| 1403 | 1417-1403 | ACAGACTCAAAATAA | 35 |
| 1404 | 1418-1404 | CACAGACTCAAAATA | 36 |
| 3528 | 3542-3528 | TATTAAATTAATGCA | 37 |
| 3529 | 3543-3529 | TTATTAAATTAATGC | 38 |
| 3530 | 3544-3530 | TTTATTAAATTAATG | 39 |
| 452 | 466-452 | GGGCATTGACTTTCA | 40 |
| 453 | 467-453 | GGGGCATTGACTTTC | 41 |
| 454 | 468-454 | TGGGGCATTGACTTT | 42 |
| 455 | 469-455 | ATGGGGCATTGACTT | 43 |
| 456 | 470-456 | TATGGGGCATTGACT | 44 |
| 457 | 471-457 | GTATGGGGCATTGAC | 45 |
| 641 | 655-641 | TGCTGGTCACATTGA | 46 |

TABLE 10
CD274 ASOs - 16mers

| Target | Target | | SEQ |
|---|---|---|---|
| start | positions | Oligo Sequence | ID |
| position | spanned | (5′ → 3′) | NO: |
| 67 | 82-67 | CAAATATCCTCATCTT | 47 |
| 68 | 83-68 | GCAAATATCCTCATCT | 48 |
| 69 | 84-69 | AGCAAATATCCTCATC | 49 |
| 70 | 85-70 | CAGCAAATATCCTCAT | 50 |
| 71 | 86-71 | ACAGCAAATATCCTCA | 51 |
| 72 | 87-72 | GACAGCAAATATCCTC | 52 |
| 1397 | 1412-1397 | CTCAAAATAAATAGGA | 53 |
| 1398 | 1413-1398 | ACTCAAAATAAATAGG | 54 |
| 1399 | 1414-1399 | GACTCAAAATAAATAG | 55 |
| 1400 | 1415-1400 | AGACTCAAAATAAATA | 56 |
| 1401 | 1416-1401 | CAGACTCAAAATAAAT | 57 |
| 1402 | 1417-1402 | ACAGACTCAAAATAAA | 58 |
| 1403 | 1418-1403 | CACAGACTCAAAATAA | 59 |
| 1404 | 1419-1404 | TCACAGACTCAAAATA | 60 |
| 1405 | 1420-1405 | CTCACAGACTCAAAAT | 61 |
| 3527 | 3542-3527 | TATTAAATTAATGCAG | 62 |
| 3528 | 3543-3528 | TTATTAAATTAATGCA | 63 |
| 3529 | 3544-3529 | TTTATTAAATTAATGC | 64 |
| 3530 | 3545-3530 | TTTTATTAAATTAATG | 65 |
| 361 | 376-361 | CTGTGATCTGAAGTGC | 66 |
| 362 | 377-362 | TCTGTGATCTGAAGTG | 67 |
| 364 | 379-364 | CATCTGTGATCTGAAG | 68 |
| 365 | 380-365 | ACATCTGTGATCTGAA | 69 |
| 430 | 445-430 | TTCGCTTGTAGTCGGC | 70 |
| 431 | 446-431 | ATTCGCTTGTAGTCGG | 71 |
| 433 | 448-433 | TAATTCGCTTGTAGTC | 72 |
| 434 | 449-434 | GTAATTCGCTTGTAGT | 73 |
| 451 | 466-451 | GGGCATTGACTTTCAC | 74 |
| 452 | 467-452 | GGGGCATTGACTTTCA | 75 |
| 453 | 468-453 | TGGGGCATTGACTTTC | 76 |
| 454 | 469-454 | ATGGGGCATTGACTTT | 77 |
| 455 | 470-455 | TATGGGGCATTGACTT | 78 |
| 456 | 471-456 | GTATGGGGCATTGACT | 79 |
| 457 | 472-457 | TGTATGGGGCATTGAC | 80 |
| 638 | 653-638 | CTGGTCACATTGAAAA | 81 |
| 639 | 654-639 | GCTGGTCACATTGAAA | 82 |
| 640 | 655-640 | TGCTGGTCACATTGAA | 83 |
| 641 | 656-641 | GTGCTGGTCACATTGA | 84 |
| 740 | 755-740 | AGTTCTGGGATGACCA | 85 |
| 743 | 758-743 | GGTAGTTCTGGGATGA | 86 |
| 1 | 16-1 | GAAGCTGCGCAGAACT | 240 |
| 97 | 112-97 | AATGCCAGTAGGTCAT | 241 |
| 121 | 136-121 | CCGTGACAGTAAATGC | 242 |
| 145 | 160-145 | CTACCACATATAGGTC | 243 |
| 169 | 184-169 | CAATTGTCATATTGCT | 244 |
| 217 | 232-217 | CAATTAGTGCAGCCAG | 245 |
| 265 | 280-265 | CTCCATGCACAAATTG | 246 |
| 289 | 304-289 | TATGCTGAACCTTCAG | 247 |
| 313 | 328-313 | GCCGGGCCCTCTGTCT | 248 |
| 529 | 544-529 | CCTCAGCCTGACATGT | 249 |
| 553 | 568-553 | AGATGACTTCGGCCTT | 250 |
| 697 | 712-697 | CTAATCTCCTAAAAGT | 251 |
| 793 | 808-793 | TGGCTCCCAGAATTAC | 252 |
| 841 | 856-841 | TTCTTAAACGGAAGAT | 253 |
| 913 | 928-913 | AATGTGTATCACTTTG | 254 |
| 937 | 952-937 | CAATGCTGGATTACGT | 255 |
| 1009 | 1024-1009 | CTCTTGTCACGCTCAG | 256 |
| 1129 | 1144-1129 | CAGGCTCCCTGTTTGA | 257 |
| 1153 | 1168-1153 | TTTGAAAGTATCAAGG | 258 |
| 1177 | 1192-1177 | AGGCGTCGATGAGCCC | 259 |
| 1201 | 1216-1201 | AGAAGTATCCTTTCTC | 260 |
| 1225 | 1240-1225 | GATTTGCTTGGAGGCT | 261 |
| 1273 | 1288-1273 | CCTCAAATTAGGGATT | 262 |
| 1345 | 1360-1345 | GAGACTCTCAGTCATG | 263 |
| 1369 | 1384-1369 | TAAATACTGTCCCGTT | 264 |
| 1465 | 1480-1465 | TCTACTACAATATATC | 265 |
| 1489 | 1504-1489 | TAGTTTGGCGACAAAA | 266 |
| 1513 | 1528-1513 | GAGCAAATCATTAAGC | 267 |
| 1561 | 1576-1561 | ATAGAGGAGACCAAGC | 268 |
| 1609 | 1624-1609 | ATGCAACCAACGGTTT | 269 |
| 1657 | 1672-1657 | AGATTAGGTCAACCAG | 270 |
| 1729 | 1744-1729 | GGCTACCACATAATTG | 271 |
| 1753 | 1768-1753 | CGATGAAATGAGATTA | 272 |
| 1777 | 1792-1777 | GTTATCACAACAGGGT | 273 |
| 1801 | 1816-1801 | TGTACGATGGGTAAAA | 274 |
| 1897 | 1912-1897 | AGCTGTAAATTGTATT | 275 |
| 1969 | 1984-1969 | GCACAGCGATTGATAT | 276 |
| 2041 | 2056-2041 | TCTCCTCATTATACTA | 277 |
| 2089 | 2104-2089 | TTATGCTATGACACTG | 278 |
| 2113 | 2128-2113 | TCGGGTTTTCCCCTCG | 279 |
| 2161 | 2176-2161 | AACCGTCCCAGACCAC | 280 |
| 2233 | 2248-2233 | GGGTTATTTTAAGTAC | 281 |
| 2305 | 2320-2305 | ATTAGATTATATGGCA | 282 |
| 2497 | 2512-2497 | AATGAAAGGCAGTAGC | 283 |
| 2713 | 2728-2713 | CTATGGGAAAGATAAC | 284 |
| 2761 | 2776-2761 | GTAGGACATATTTAAC | 285 |
| 2785 | 2800-2785 | AATGGTGGTTGTCTAA | 286 |
| 2809 | 2824-2809 | CTGTCCTAGAGCAAAT | 287 |
| 2881 | 2896-2881 | GACTAGATTGACTCAG | 288 |
| 2905 | 2920-2905 | GTTAATAATAAGATTG | 289 |
| 2977 | 2992-2977 | AGCATCAAAGTGAAGT | 290 |
| 3025 | 3040-3025 | AGGTACACTGCCGGAA | 291 |
| 3073 | 3088-3073 | TCAAGCACAACGAATG | 292 |
| 3097 | 3112-3097 | TGACAGCTGGTGGCAT | 293 |
| 3121 | 3136-3121 | CCTCTTAGGAGGGCTG | 294 |
| 3145 | 3160-3145 | CTGAATCTCGAAACCT | 295 |
| 3265 | 3280-3265 | GAGCTCTGTTGGAGAC | 296 |
| 3313 | 3328-3313 | TCACACCAATTACTGT | 297 |
| 3385 | 3400-3385 | AGGAATAGACTGAGTA | 298 |
| 3433 | 3448-3433 | GGATAAAGTGCCTTAC | 299 |
| 3457 | 3472-3457 | TTACGATGAAACATGA | 300 |
| 3577 | 3592-3577 | AGAAAATGGACATGCT | 301 |

TABLE 11
CD274 ASOs - 17mers

| Target | Target | | SEQ |
|---|---|---|---|
| start | positions | Oligo Sequence | ID |
| position | spanned | (5′ → 3′) | NO: |
| 67 | 83-67 | GCAAATATCCTCATCTT | 87 |
| 68 | 84-68 | AGCAAATATCCTCATCT | 88 |
| 69 | 85-69 | CAGCAAATATCCTCATC | 89 |
| 70 | 86-70 | ACAGCAAATATCCTCAT | 90 |
| 71 | 87-71 | GACAGCAAATATCCTCA | 91 |
| 1397 | 1413-1397 | ACTCAAAATAAATAGGA | 92 |
| 1398 | 1414-1398 | GACTCAAAATAAATAGG | 93 |
| 1399 | 1415-1399 | AGACTCAAAATAAATAG | 94 |
| 1400 | 1416-1400 | CAGACTCAAAATAAATA | 95 |
| 1401 | 1417-1401 | ACAGACTCAAAATAAAT | 96 |
| 1402 | 1418-1402 | CACAGACTCAAAATAAA | 97 |
| 1403 | 1419-1403 | TCACAGACTCAAAATAA | 98 |
| 1404 | 1420-1404 | CTCACAGACTCAAAATA | 99 |
| 3527 | 3543-3527 | TTATTAAATTAATGCAG | 100 |
| 3528 | 3544-3528 | TTTATTAAATTAATGCA | 101 |
| 3529 | 3545-3529 | TTTTATTAAATTAATGC | 102 |
| 361 | 377-361 | TCTGTGATCTGAAGTGC | 103 |
| 364 | 380-364 | ACATCTGTGATCTGAAG | 104 |
| 430 | 446-430 | ATTCGCTTGTAGTCGGC | 105 |
| 433 | 449-433 | GTAATTCGCTTGTAGTC | 106 |
| 451 | 467-451 | GGGGCATTGACTTTCAC | 107 |
| 452 | 468-452 | TGGGGCATTGACTTTCA | 108 |
| 453 | 469-453 | ATGGGGCATTGACTTTC | 109 |
| 454 | 470-454 | TATGGGGCATTGACTTT | 110 |
| 455 | 471-455 | GTATGGGGCATTGACTT | 111 |
| 456 | 472-456 | TGTATGGGGCATTGACT | 112 |
| 638 | 654-638 | GCTGGTCACATTGAAAA | 113 |
| 639 | 655-639 | TGCTGGTCACATTGAAA | 114 |

TABLE 12
CD274 ASOs - 18mers

| Target | Target | | SEQ |
|---|---|---|---|
| start | positions | Oligo Sequence | ID |
| position | spanned | (5′ → 3′) | NO: |
| 66 | 83-66 | GCAAATATCCTCATCTTT | 115 |
| 67 | 84-67 | AGCAAATATCCTCATCTT | 116 |
| 68 | 85-68 | CAGCAAATATCCTCATCT | 117 |
| 69 | 86-69 | ACAGCAAATATCCTCATC | 118 |
| 70 | 87-70 | GACAGCAAATATCCTCAT | 119 |
| 71 | 88-71 | AGACAGCAAATATCCTCA | 120 |
| 72 | 89-72 | AAGACAGCAAATATCCTC | 121 |
| 1396 | 1413-1396 | ACTCAAAATAAATAGGAA | 122 |
| 1397 | 1414-1397 | GACTCAAAATAAATAGGA | 123 |
| 1398 | 1415-1398 | AGACTCAAAATAAATAGG | 124 |
| 1399 | 1416-1399 | CAGACTCAAAATAAATAG | 125 |
| 1400 | 1417-1400 | ACAGACTCAAAATAAATA | 126 |
| 1401 | 1418-1401 | CACAGACTCAAAATAAAT | 127 |
| 1402 | 1419-1402 | TCACAGACTCAAAATAAA | 128 |
| 1403 | 1420-1403 | CTCACAGACTCAAAATAA | 129 |
| 1404 | 1421-1404 | CCTCACAGACTCAAAATA | 130 |
| 1405 | 1422-1405 | ACCTCACAGACTCAAAAT | 131 |
| 3237 | 3254-3237 | CAGCCTTGACATGTGGCA | 132 |
| 3526 | 3543-3526 | TTATTAAATTAATGCAGG | 133 |
| 3527 | 3544-3527 | TTTATTAAATTAATGCAG | 134 |
| 3528 | 3545-3528 | TTTTATTAAATTAATGCA | 135 |
| 3529 | 3546-3529 | ATTTTATTAAATTAATGC | 136 |
| 360 | 377-360 | TCTGTGATCTGAAGTGCA | 137 |
| 361 | 378-361 | ATCTGTGATCTGAAGTGC | 138 |
| 362 | 379-362 | CATCTGTGATCTGAAGTG | 139 |
| 363 | 380-363 | ACATCTGTGATCTGAAGT | 140 |
| 364 | 381-364 | CACATCTGTGATCTGAAG | 141 |
| 429 | 446-429 | ATTCGCTTGTAGTCGGCA | 142 |
| 430 | 447-430 | AATTCGCTTGTAGTCGGC | 143 |
| 431 | 448-431 | TAATTCGCTTGTAGTCGG | 144 |
| 432 | 449-432 | GTAATTCGCTTGTAGTCG | 145 |
| 433 | 450-433 | AGTAATTCGCTTGTAGTC | 146 |
| 448 | 465-448 | GGCATTGACTTTCACAGT | 147 |
| 449 | 466-449 | GGGCATTGACTTTCACAG | 148 |
| 450 | 467-450 | GGGGCATTGACTTTCACA | 149 |
| 451 | 468-451 | TGGGGCATTGACTTTCAC | 150 |
| 452 | 469-452 | ATGGGGCATTGACTTTCA | 151 |
| 453 | 470-453 | TATGGGGCATTGACTTTC | 152 |
| 454 | 471-454 | GTATGGGGCATTGACTTT | 153 |
| 455 | 472-455 | TGTATGGGGCATTGACTT | 154 |
| 456 | 473-456 | TTGTATGGGGCATTGACT | 155 |
| 457 | 474-457 | GTTGTATGGGGCATTGAC | 156 |
| 458 | 475-458 | TGTTGTATGGGGCATTGA | 157 |
| 637 | 654-637 | GCTGGTCACATTGAAAAG | 158 |
| 638 | 655-638 | TGCTGGTCACATTGAAAA | 159 |
| 639 | 656-639 | GTGCTGGTCACATTGAAA | 160 |
| 641 | 658-641 | GTGTGCTGGTCACATTGA | 161 |
| 642 | 659-642 | AGTGTGCTGGTCACATTG | 162 |
| 740 | 757-740 | GTAGTTCTGGGATGACCA | 163 |
| 741 | 758-741 | GGTAGTTCTGGGATGACC | 164 |
| 742 | 759-742 | AGGTAGTTCTGGGATGAC | 165 |

TABLE 13
CD274 ASOs - 19mers

| Target | Target | | SEQ |
|---|---|---|---|
| start | positions | Oligo Sequence | ID |
| position | spanned | (5′ → 3′) | NO: |
| 66 | 84-66 | AGCAAATATCCTCATCTTT | 166 |
| 67 | 85-67 | CAGCAAATATCCTCATCTT | 167 |
| 68 | 86-68 | ACAGCAAATATCCTCATCT | 168 |
| 69 | 87-69 | GACAGCAAATATCCTCATC | 169 |
| 70 | 88-70 | AGACAGCAAATATCCTCAT | 170 |
| 71 | 89-71 | AAGACAGCAAATATCCTCA | 171 |
| 1396 | 1414-1396 | GACTCAAAATAAATAGGAA | 172 |
| 1397 | 1415-1397 | AGACTCAAAATAAATAGGA | 173 |
| 1398 | 1416-1398 | CAGACTCAAAATAAATAGG | 174 |
| 1399 | 1417-1399 | ACAGACTCAAAATAAATAG | 175 |
| 1400 | 1418-1400 | CACAGACTCAAAATAAATA | 176 |
| 1401 | 1419-1401 | TCACAGACTCAAAATAAAT | 177 |
| 1402 | 1420-1402 | CTCACAGACTCAAAATAAA | 178 |
| 1403 | 1421-1403 | CCTCACAGACTCAAAATAA | 179 |
| 1404 | 1422-1404 | ACCTCACAGACTCAAAATA | 180 |
| 3526 | 3544-3526 | TTTATTAAATTAATGCAGG | 181 |
| 3527 | 3545-3527 | TTTTATTAAATTAATGCAG | 182 |
| 3528 | 3546-3528 | ATTTTATTAAATTAATGCA | 183 |
| 360 | 378-360 | ATCTGTGATCTGAAGTGCA | 184 |
| 361 | 379-361 | CATCTGTGATCTGAAGTGC | 185 |
| 362 | 380-362 | ACATCTGTGATCTGAAGTG | 186 |
| 429 | 447-429 | AATTCGCTTGTAGTCGGCA | 187 |
| 430 | 448-430 | TAATTCGCTTGTAGTCGGC | 188 |
| 431 | 449-431 | GTAATTCGCTTGTAGTCGG | 189 |
| 448 | 466-448 | GGGCATTGACTTTCACAGT | 190 |
| 449 | 467-449 | GGGGCATTGACTTTCACAG | 191 |
| 450 | 468-450 | TGGGGCATTGACTTTCACA | 192 |
| 451 | 469-451 | ATGGGGCATTGACTTTCAC | 193 |
| 452 | 470-452 | TATGGGGCATTGACTTTCA | 194 |
| 453 | 471-453 | GTATGGGGCATTGACTTTC | 195 |
| 454 | 472-454 | TGTATGGGGCATTGACTTT | 196 |
| 455 | 473-455 | TTGTATGGGGCATTGACTT | 197 |
| 456 | 474-456 | GTTGTATGGGGCATTGACT | 198 |
| 457 | 475-457 | TGTTGTATGGGGCATTGAC | 199 |
| 637 | 655-637 | TGCTGGTCACATTGAAAAG | 200 |
| 638 | 656-638 | GTGCTGGTCACATTGAAAA | 201 |
| 641 | 659-641 | AGTGTGCTGGTCACATTGA | 202 |
| 740 | 758-740 | GGTAGTTCTGGGATGACCA | 203 |
| 741 | 759-741 | AGGTAGTTCTGGGATGACC | 204 |

TABLE 14
CD274 ASOs - 20mers

| Target | Target | | SEQ |
|---|---|---|---|
| start | positions | Oligo Sequence | ID |
| position | spanned | (5′ → 3′) | NO: |
| 65 | 84-65 | AGCAAATATCCTCATCTTTC | 205 |
| 66 | 85-66 | CAGCAAATATCCTCATCTTT | 206 |
| 67 | 86-67 | ACAGCAAATATCCTCATCTT | 207 |
| 68 | 87-68 | GACAGCAAATATCCTCATCT | 208 |
| 70 | 89-70 | AAGACAGCAAATATCCTCAT | 209 |
| 71 | 90-71 | AAAGACAGCAAATATCCTCA | 210 |
| 1396 | 1415-1396 | AGACTCAAAATAAATAGGAA | 211 |
| 1397 | 1416-1397 | CAGACTCAAAATAAATAGGA | 212 |
| 1398 | 1417-1398 | ACAGACTCAAAATAAATAGG | 213 |
| 1399 | 1418-1399 | CACAGACTCAAAATAAATAG | 214 |
| 1400 | 1419-1400 | TCACAGACTCAAAATAAATA | 215 |
| 1401 | 1420-1401 | CTCACAGACTCAAAATAAAT | 216 |
| 1402 | 1421-1402 | CCTCACAGACTCAAAATAAA | 217 |
| 1403 | 1422-1403 | ACCTCACAGACTCAAAATAA | 218 |
| 1404 | 1423-1404 | GACCTCACAGACTCAAAATA | 219 |
| 359 | 378-359 | ATCTGTGATCTGAAGTGCAG | 220 |
| 360 | 379-360 | CATCTGTGATCTGAAGTGCA | 221 |
| 361 | 380-361 | ACATCTGTGATCTGAAGTGC | 222 |
| 428 | 447-428 | AATTCGCTTGTAGTCGGCAC | 223 |
| 429 | 448-429 | TAATTCGCTTGTAGTCGGCA | 224 |
| 430 | 449-430 | GTAATTCGCTTGTAGTCGGC | 225 |
| 448 | 467-448 | GGGGCATTGACTTTCACAGT | 226 |
| 449 | 468-449 | TGGGGCATTGACTTTCACAG | 227 |
| 450 | 469-450 | ATGGGGCATTGACTTTCACA | 228 |
| 451 | 470-451 | TATGGGGCATTGACTTTCAC | 229 |
| 452 | 471-452 | GTATGGGGCATTGACTTTCA | 230 |
| 453 | 472-453 | TGTATGGGGCATTGACTTTC | 231 |
| 454 | 473-454 | TTGTATGGGGCATTGACTTT | 232 |
| 455 | 474-455 | GTTGTATGGGGCATTGACTT | 233 |
| 456 | 475-456 | TGTTGTATGGGGCATTGACT | 234 |
| 457 | 476-457 | TTGTTGTATGGGGCATTGAC | 235 |
| 636 | 655-636 | TGCTGGTCACATTGAAAAGC | 236 |
| 637 | 656-637 | GTGCTGGTCACATTGAAAAG | 237 |
| 641 | 660-641 | CAGTGTGCTGGTCACATTGA | 238 |
| 740 | 759-740 | AGGTAGTTCTGGGATGACCA | 239 |

### Example 3: Treatment of Cancer Using CD274 ASOs

A human patient presents with a cancer, such as a hepatocellular carcinoma (HCC). The cancer is a non-metastatic or metastatic cancer. In the case of HCC, the patient may also have another liver condition, such as fibrosis, cirrhosis, non-alcoholic liver disease, hepatitis, hepatitis B, or hepatitis C. An effective amount of a CD274 ASO or a pharmaceutical composition comprising an effective amount of a CD274 ASO is administered to the patient parenterally. The CD274 ASO is selected from the group consisting of SEQ ID NOs: 2-301. The CD274 ASO can optionally have any of the modifications to individual nucleobases, sugars, linkages, nucleosides, or nucleotides as described herein. The CD274 ASO can also optionally have a covalently conjugated targeting moiety to improve selectivity to tumor and/or liver tissue. The CD274 ASO can be constructed of deoxyribose sugars (DNA nucleotides), ribose sugars (RNA nucleotides) or any combination thereof. The CD274 ASO can be constructed of unmodified nucleotides or modified nucleotides or any combination thereof and optionally can be a gapmer, mixmer, or blockmer. The CD274 ASO or pharmaceutical composition comprising the CD274 ASO can optionally be administered as a combination therapy with another anti-neoplastic compound or therapy.

Following administration of an effective amount of the CD274 ASO or the pharmaceutical composition comprising an effective amount of the CD274 ASO, the cancer is reduced or eliminated.

### Example 4: Treatment of Hepatitis B Using CD274 ASOs

A human patient presents with a hepatitis B infection. The hepatitis B infection is acute or chronic. The hepatitis B infection may also be coincidental with a hepatitis D infection. The patient may also have another liver conditions, such as fibrosis, cirrhosis, non-alcoholic liver disease, or HCC. An effective amount of a CD274 ASO or a pharmaceutical composition comprising an effective amount of a CD274 ASO is administered to the patient parenterally. The CD274 ASO is selected from the group consisting of SEQ ID NOs: 2-301. The CD274 ASO can optionally have any of the modifications to individual nucleobases, sugars, linkages, nucleosides, or nucleotides as described herein. The CD274 ASO can also optionally have a covalently conjugated targeting moiety to improve selectivity to liver tissue. The CD274 ASO can be constructed of deoxyribose sugars (DNA nucleotides), ribose sugars (RNA nucleotides) or any combination thereof. The CD274 ASO can be constructed of unmodified nucleotides or modified nucleotides or any combination thereof and optionally can be a gapmer, mixmer, or blockmer. The CD274 ASO or pharmaceutical composition comprising the CD274 ASO can optionally be administered as a combination therapy with another antiviral medication.

Following administration of an effective amount of the CD274 ASO or the pharmaceutical composition comprising an effective amount of the CD274 ASO, the hepatitis B infection (and optionally, hepatitis D infection) is reduced or eliminated.

### Example 5: Treatment of Hepatocellular Carcinoma Cells Using ASOs

Human hepatocellular carcinoma cells (SNU-387) were seeded at 30,000 cells/well in a 96-well plate. The ASOs, including any of SEQ ID NOs: 2-301, were transfected with Lipofectamine RNAiMax (Life Technologies) in the seeded SNU-387 cells. The ASOs included any of the modifications described herein, including modification of individual nucleobases, sugars, linkages, nucleosides, or nucleotides. ASOs were screened at two concentrations, 100 nM and 1 nM or 50 nM and 1 nm. Active ASOs were further screened to obtain EC50 values via dose response curves. Selected ASO subsets were selected having greater than 50% K.D. at 100 nM, and limited toxicity.

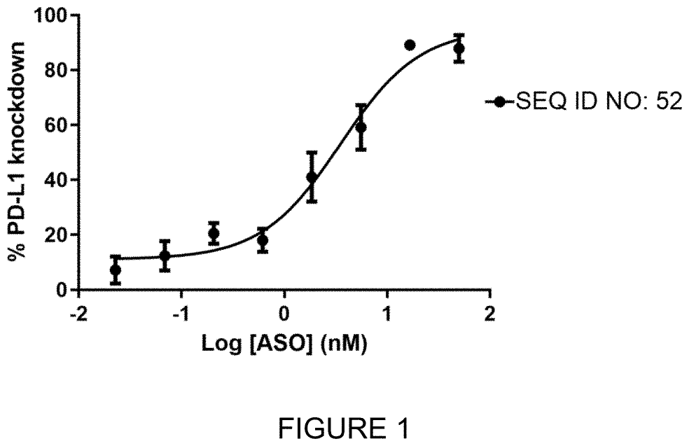

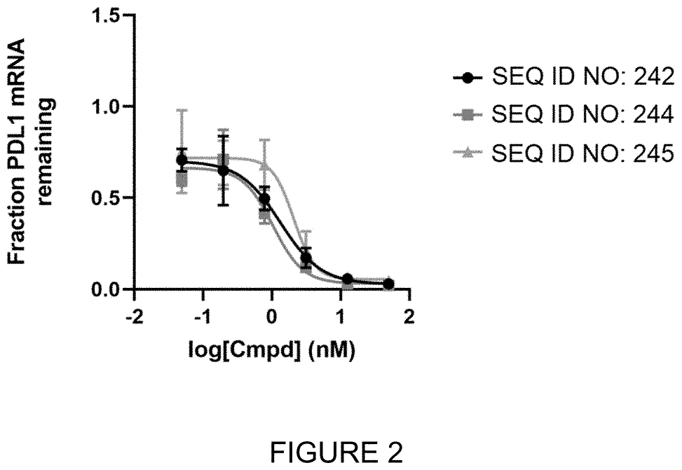

For dose response curves, a 3-fold dilution series of ASO (top dose 50 nM; six or eight concentrations tested total) was tested. The cells were harvested 48 hours after transfection, and RNA was extracted with RNeasy Kit (Qiagen). RT-qPCR was performed to assess PD-L1 gene knockdown. Cell viability was assessed at 48 hours post transfection using CCK8 assay. Data was fit with fitting software using a four-parameter dose response equation. Table 15 provides representative EC50 and CC50 values for selected ASOs, each of which have a LNA-DNA-LNA (3-10-3) modification with each linkage between the nucleosides a phosphorothioate (a PS backbone), and with all cytosines a (5m)C. Tables 16 and 17 depict percent reduction of PD-L1 gene for select ASOs, each of which have a LNA-DNA-LNA (3-10-3) modification. FIG. 1 depicts the percent PD-L1 knockdown as a function of log concentration of ASO in nM. FIG. 2 depicts the fraction of PD-L1 mRNA remaining for three exemplary ASOs as a function of log concentration of ASO in nM.

TABLE 15
EC50 and CC50 for select ASOs

| SEQ ID | EC50 | CC50 |
|---|---|---|
| NO: | (nM) | (nM) |
| 55 | C | X |
| 48 | C | X |
| 52 | B | X |
| 69 | C | X |
| 84 | B | X |
| 242 | B | X |
| 243 | A | X |
| 245 | B | X |
| 262 | C | X |
| 264 | C | X |
| 267 | B | X |
| 272 | B | X |
| 275 | A | X |
| 276 | B | X |
| 282 | B | X |
| 283 | B | X |

A ≤ 1 nM; B > 1-5 nM; C > 5-10 nM
X > 50 nM;Y ≤ 50 nM

TABLE 16
Percent Reduction of PD-L1 Gene with select ASOs

| SEQ | % Reduction | % Reduction of | |
|---|---|---|---|
| ID | of PD-L1 gene | PD-L1 gene | CC50 |
| NO: | at 100 nM | at 1 nM | (nM) |
| 50 | B | D | X |
| 75 | D | D | Y |
| 76 | B | D | Y |
| 77 | A | D | Y |
| 78 | A | D | X |
| 79 | A | D | X |
| 55 | A | D | X |
| 56 | C | D | X |
| 57 | C | D | X |
| 58 | C | D | X |
| 59 | C | D | X |
| 63 | C | D | X |
| 64 | C | D | X |
| 47 | C | D | X |
| 48 | A | D | X |
| 49 | A | D | X |
| 51 | A | D | X |
| 52 | A | D | X |
| 66 | A | D | Y |
| 67 | A | D | X |
| 68 | A | D | X |
| 69 | A | D | X |
| 70 | A | D | X |
| 71 | A | D | X |
| 72 | A | D | X |
| 73 | A | D | X |
| 74 | B | D | X |
| 80 | A | D | Y |
| 81 | B | D | Y |
| 82 | B | D | Y |
| 83 | A | D | X |
| 84 | A | D | X |
| 85 | B | D | Y |
| 86 | B | D | Y |
| 53 | B | D | X |
| 54 | B | D | X |
| 60 | C | D | X |
| 61 | C | D | X |
| 62 | C | D | X |
| 65 | D | D | X |

A > 75%-100%; B > 50%-75%; C > 25%-50%; D = 0%-25%
X > 100 nM; Y ≤ 100 nM

TABLE 17
Percent Reduction of PD-L1 Gene with select ASOs

| SEQ | % Reduction | % Reduction of | |
|---|---|---|---|
| ID | of PD-L1 gene | PD-L1 gene | CC50 |
| NO: | at 50 nM | at 1 nM | (nM) |
| 240 | D | C | Y |
| 241 | D | D | X |
| 242 | A | C | X |
| 243 | D | D | X |
| 244 | B | C | X |
| 245 | B | B | X |
| 246 | C | D | X |
| 247 | C | C | X |
| 248 | D | C | Y |
| 249 | D | D | Y |
| 250 | D | C | Y |
| 251 | C | D | X |
| 252 | D | C | Y |
| 253 | C | C | X |
| 254 | D | D | X |
| 255 | D | D | Y |
| 256 | D | D | Y |
| 257 | D | D | X |
| 258 | C | D | X |
| 259 | C | C | X |
| 260 | C | C | X |
| 261 | D | D | Y |
| 262 | C | D | X |
| 263 | C | C | X |
| 264 | C | D | X |
| 265 | C | C | X |
| 266 | D | B | X |
| 267 | A | D | X |
| 268 | A | B | X |
| 269 | B | C | X |
| 270 | A | B | X |
| 271 | B | C | X |
| 272 | A | D | X |
| 273 | B | B | X |
| 274 | B | C | X |
| 275 | A | D | X |
| 276 | A | C | X |
| 277 | B | B | X |
| 278 | A | D | Y |
| 279 | B | C | X |
| 280 | B | C | X |
| 281 | B | D | X |
| 282 | A | C | X |
| 283 | B | D | X |
| 284 | C | D | X |
| 285 | A | D | X |
| 286 | B | C | X |
| 288 | C | D | X |
| 289 | B | D | X |
| 290 | B | D | X |
| 291 | D | D | X |
| 292 | D | D | Y |
| 293 | A | C | X |
| 294 | C | D | X |
| 295 | B | C | Y |
| 296 | B | C | X |
| 297 | C | C | X |
| 298 | B | D | X |
| 299 | A | D | X |
| 300 | D | D | X |
| 301 | B | D | X |
| 287 | D | D | X |

A > 75%-100%; B > 50%-75%; C > 25%-50%; D = 0-25%
X > 50 nM; Y ≤ 50 nM

The example ASOs, including the example sequences and example modifications as described in the examples, are intended as exemplary sequences and modifications. However, it is to be understood that the disclosure relates to any ASO sequence as set forth herein, having any modification or combination of modifications as set forth herein may be implemented in the examples.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

1. U.S. 2017/0283496
2. Akinleye, A & Rasool Z. Immune Checkpoint Inhibitors of PD-L1 as Cancer Therapeutics. J. Hematol. Oncol. (2019) 12(1):92.
3. Wu, Y et al. PD-L1 Distribution and Perspective for Cancer Immunotherapy—Blockade, Knockdown, or Inhibition. Front. Immunol. (2019) 10:2022.
4. Sun, C et al. Regulation and Function of the PD-L1 Checkpoint. Immunity. (2018) 48(3):434-452.
5. Schönrich, G & Raferty M J. The PD-1/PD-L1 Axis and Virus Infections: A Delicate Balance. Front. Cell. Infect. Microbiol. (2019) 9:207
6. Østergaard, M E et al. Fluorinated Nucleotide Modifications Modulate Allele Selectivity of SNP-Targeting Antisense Oligonucleotides. Mol. Ther. Nucleic Acids. (2017) 7:20-30.
7. Di Fusco, D et al. Antisense Oligonucleotide: Basic Concepts and Therapeutic Application in Inflammatory Bowel Disease. Front Pharmacol. (2019) 10:305.
8. Wurster, C D & Ludolph A C. Antisense Oligonucleotides in Neurological Disorders. Ther. Adv. Neurol. Disord. (2018) 11:1-19.
9. Balsitis S et al. Safety and Efficacy of Anti-PD-L1 Therapy in the Woodchuck Model of HBV Infection. (2018) 13(2):1-23.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. An antisense oligonucleotide (ASO) that targets human CD274 mRNA, wherein the ASO comprises 14 to 20 nucleotides selected from the group consisting of unmodified nucleotides and modified nucleosides,
wherein each of the modified nucleosides (a) contains a modified sugar, (b) contains a modified nucleobase or is abasic, or (c) both contains a modified sugar and contains a modified nucleobase or is abasic;
wherein each linkage between the nucleosides is a phosphorothioate, phosphodiester, phosphoramidate, thiophosphoramidate, methylphosphate, methylphosphonate, phosphonoacetate, boranophosphate, or any combination thereof; and
wherein the ASO is at least 85% complementary to a fragment of human CD274 mRNA.

2. The ASO of claim 1, wherein the ASO comprises:
(a) zero nucleotide mismatches if the ASO is 14 or 15 nucleotides in length;
(b) up to one nucleotide mismatch if the ASO is m nucleotides in length at position 1, 2, 3, m-2, m-1, or m, wherein m is 16 or 17;
(c) up to two nucleotide mismatches if the ASO is m nucleotides in length at position 1, 2, 3, 4, m-3, m-2, m-1, or m, wherein m is 18 or 19; or
(d) up to two nucleotide mismatches if the ASO is 20 nucleotides in length at position 1, 2, 3, 4, 5, 16, 17, 18, 19, or 20.

3. The ASO of claim 1, wherein the ASO has a sequence as set forth in any one of SEQ ID NOs: 2-301.

4. The ASO of claim 1, wherein the ASO has 14 nt.

5. The ASO of claim 1, wherein the ASO has 15 nt.

6. The ASO of claim 1, wherein the ASO has 16 nt.

7. The ASO of claim 1, wherein the ASO has 17 nt.

8. The ASO of claim 1, wherein the ASO has 18 nt.

9. The ASO of claim 1, wherein the ASO has 19 nt.

10. The ASO of claim 1, wherein the ASO has 20 nt.

11. The ASO of claim 1, wherein the modified sugar is selected from the group consisting of 2′-OMe, 2′-F, 2′-MOE, 2′-araF, 2′-araOH, 2′-OEt, 2′-O-alkyl, LNA, scpBNA, AmNA, cEt, and ENA.

12. The ASO of claim 1, wherein the modified nucleobase is selected from the group consisting of 5-OH—C, 2S-T, 8-NH2-A, 8-NH2-G, and 5-methyl-C.

13. The ASO of claim 1, further comprising a targeting or lipophilic moiety.

14. The ASO of claim 13, wherein the targeting or lipophilic moiety is conjugated to the ASO at the 5′ end, 3′ end, or both.

15. The ASO of claim 13, wherein the targeting or lipophilic moiety is GalNAc, folic acid, cholesterol, tocopherol, palmitate, or a long chain fatty acid.

16. The ASO according to claim 1, wherein the ASO is a gapmer, mixmer, or blockmer.

17. The ASO of claim 1, wherein the modified nucleosides are selected from the groin consisting of: 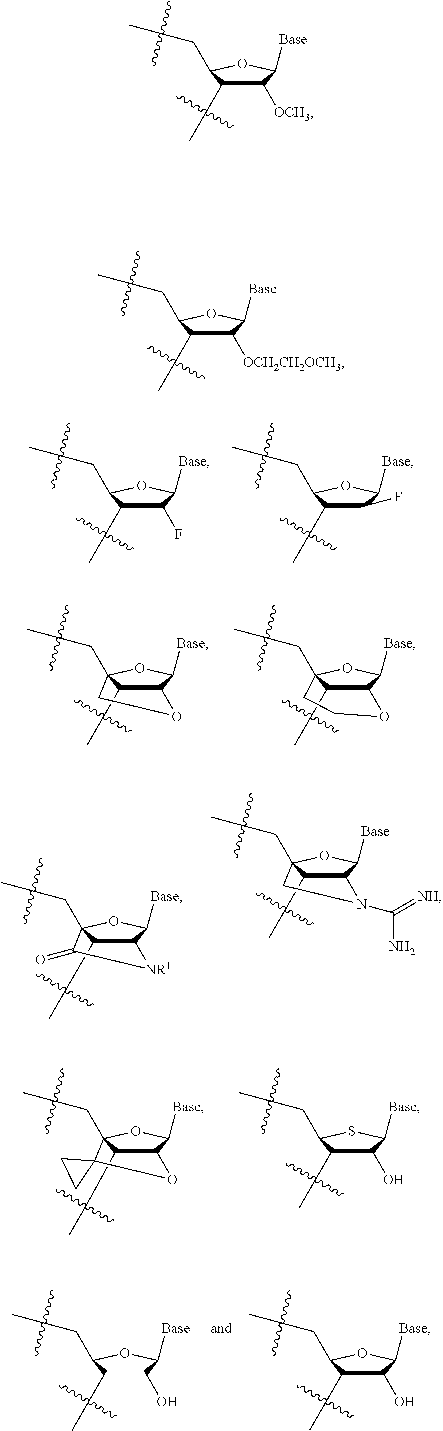
wherein R¹ is hydrogen or C₁₋₇ alkyl.

18. The ASO of claim 17, wherein the Base is selected from the group consisting of adenine, guanine, cytosine, thymine, uracil, pseudouracil, 2-thio-uracil, dihydrouracil, 5-bromo-uracil, 5-iodo-uracil, 5′-methyl-cytosine, 7-deazapurine, 2,6-diaminopurine, inosine, phenoxazine and

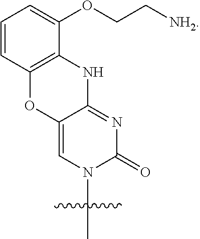 [SMILES: *n1cc2c(nc1=O)Nc1c(OCCN)cccc1O2]

19. The ASO of claim 18, wherein the modified nucleobase is selected from the group consisting of pseudouracil, 2-thio-uracil, dihydrouracil, 5-bromo-uracil, 5-iodo-uracil, 5-methyl-cytosine, 7-deazapurine, 2,6-diaminopurine, inosine, phenoxazine and

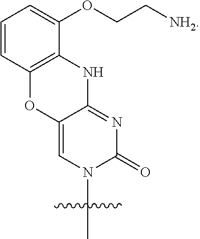 [SMILES: *n1cc2c(nc1=O)Nc1c(OCCN)cccc1O2]

20. A pharmaceutical composition comprising an effective amount of the ASO according to claim 1 and a pharmaceutically acceptable carrier, diluent, excipient, or combination thereof.

21. (canceled)

22. (canceled)

23. (canceled)

24. (canceled)

25. A method for treating hepatitis B in a subject comprising administering to the subject in need thereof an effective amount of an ASO of claim 1.

26. A method for treating hepatocellular carcinoma (HCC) in a subject comprising administering to the subject in need thereof an effective amount of an ASO of claim 1.

27. The method of claim 25, further comprising administering surgery, radiation therapy, chemotherapy, targeted therapy, immunotherapy, hormonal therapy, or antiviral therapy.


SEQUENCE LISTING (301 sequences)

SEQ ID NO: 1 - DNA, 3634 bp, Artificial Sequence
  Human CD274
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 2 - DNA, 14 bp, Artificial Sequence
  Synthetic Oligonucleotide
gcaaatatcctcat

SEQ ID NO: 3 - DNA, 14 bp, Artificial Sequence
  Synthetic Oligonucleotide
agcaaatatcctca

SEQ ID NO: 4 - DNA, 14 bp, Artificial Sequence
  Synthetic Oligonucleotide
cagcaaatatcctc

SEQ ID NO: 5 - DNA, 14 bp, Artificial Sequence
  Synthetic Oligonucleotide
ctcaaaataaatag

SEQ ID NO: 6 - DNA, 14 bp, Artificial Sequence
  Synthetic Oligonucleotide
actcaaaataaata

SEQ ID NO: 7 - DNA, 14 bp, Artificial Sequence
  Synthetic Oligonucleotide
gactcaaaataaat

SEQ ID NO: 8 - DNA, 14 bp, Artificial Sequence
  Synthetic Oligonucleotide
agactcaaaataaa

SEQ ID NO: 9 - DNA, 14 bp, Artificial Sequence
  Synthetic Oligonucleotide
cagactcaaaataa

SEQ ID NO: 10 - DNA, 14 bp, Artificial Sequence
  Synthetic Oligonucleotide
acagactcaaaata

SEQ ID NO: 11 - DNA, 14 bp, Artificial Sequence
  Synthetic Oligonucleotide
cacagactcaaaat

SEQ ID NO: 12 - DNA, 14 bp, Artificial Sequence
  Synthetic Oligonucleotide
catttaaatagaaa

SEQ ID NO: 13 - DNA, 14 bp, Artificial Sequence
  Synthetic Oligonucleotide
aataaataaaaata

SEQ ID NO: 14 - DNA, 14 bp, Artificial Sequence
  Synthetic Oligonucleotide
attaaattaatgca

SEQ ID NO: 15 - DNA, 14 bp, Artificial Sequence
  Synthetic Oligonucleotide
tattaaattaatgc

SEQ ID NO: 16 - DNA, 14 bp, Artificial Sequence
  Synthetic Oligonucleotide
ttattaaattaatg

SEQ ID NO: 17 - DNA, 14 bp, Artificial Sequence
  Synthetic Oligonucleotide
tttattaaattaat

SEQ ID NO: 18 - DNA, 14 bp, Artificial Sequence
  Synthetic Oligonucleotide
tctgtgatctgaag

SEQ ID NO: 19 - DNA, 14 bp, Artificial Sequence
  Synthetic Oligonucleotide
attcgcttgtagtc

SEQ ID NO: 20 - DNA, 14 bp, Artificial Sequence
  Synthetic Oligonucleotide
ggcattgactttca

SEQ ID NO: 21 - DNA, 14 bp, Artificial Sequence
  Synthetic Oligonucleotide
gggcattgactttc

SEQ ID NO: 22 - DNA, 14 bp, Artificial Sequence
  Synthetic Oligonucleotide
ggggcattgacttt

SEQ ID NO: 23 - DNA, 14 bp, Artificial Sequence
  Synthetic Oligonucleotide
tggggcattgactt

SEQ ID NO: 24 - DNA, 14 bp, Artificial Sequence
  Synthetic Oligonucleotide
atggggcattgact

SEQ ID NO: 25 - DNA, 14 bp, Artificial Sequence
  Synthetic Oligonucleotide
tatggggcattgac

SEQ ID NO: 26 - DNA, 14 bp, Artificial Sequence
  Synthetic Oligonucleotide
gtatggggcattga

SEQ ID NO: 27 - DNA, 14 bp, Artificial Sequence
  Synthetic Oligonucleotide
gctggtcacattga

SEQ ID NO: 28 - DNA, 14 bp, Artificial Sequence
  Synthetic Oligonucleotide
tgctggtcacattg

SEQ ID NO: 29 - DNA, 15 bp, Artificial Sequence
  Synthetic Oligonucleotide
agcaaatatcctcat

SEQ ID NO: 30 - DNA, 15 bp, Artificial Sequence
  Synthetic Oligonucleotide
cagcaaatatcctca

SEQ ID NO: 31 - DNA, 15 bp, Artificial Sequence
  Synthetic Oligonucleotide
actcaaaataaatag

SEQ ID NO: 32 - DNA, 15 bp, Artificial Sequence
  Synthetic Oligonucleotide
gactcaaaataaata

SEQ ID NO: 33 - DNA, 15 bp, Artificial Sequence
  Synthetic Oligonucleotide
agactcaaaataaat

SEQ ID NO: 34 - DNA, 15 bp, Artificial Sequence
  Synthetic Oligonucleotide
cagactcaaaataaa

SEQ ID NO: 35 - DNA, 15 bp, Artificial Sequence
  Synthetic Oligonucleotide
acagactcaaaataa

SEQ ID NO: 36 - DNA, 15 bp, Artificial Sequence
  Synthetic Oligonucleotide
cacagactcaaaata

SEQ ID NO: 37 - DNA, 15 bp, Artificial Sequence
  Synthetic Oligonucleotide
tattaaattaatgca

SEQ ID NO: 38 - DNA, 15 bp, Artificial Sequence
  Synthetic Oligonucleotide
ttattaaattaatgc

SEQ ID NO: 39 - DNA, 15 bp, Artificial Sequence
  Synthetic Oligonucleotide
tttattaaattaatg

SEQ ID NO: 40 - DNA, 15 bp, Artificial Sequence
  Synthetic Oligonucleotide
gggcattgactttca

SEQ ID NO: 41 - DNA, 15 bp, Artificial Sequence
  Synthetic Oligonucleotide
ggggcattgactttc

SEQ ID NO: 42 - DNA, 15 bp, Artificial Sequence
  Synthetic Oligonucleotide
tggggcattgacttt

SEQ ID NO: 43 - DNA, 15 bp, Artificial Sequence
  Synthetic Oligonucleotide
atggggcattgactt

SEQ ID NO: 44 - DNA, 15 bp, Artificial Sequence
  Synthetic Oligonucleotide
tatggggcattgact

SEQ ID NO: 45 - DNA, 15 bp, Artificial Sequence
  Synthetic Oligonucleotide
gtatggggcattgac

SEQ ID NO: 46 - DNA, 15 bp, Artificial Sequence
  Synthetic Oligonucleotide
tgctggtcacattga

SEQ ID NO: 47 - DNA, 16 bp, Artificial Sequence
  Synthetic Oligonucleotide
caaatatcctcatctt

SEQ ID NO: 48 - DNA, 16 bp, Artificial Sequence
  Synthetic Oligonucleotide
gcaaatatcctcatct

SEQ ID NO: 49 - DNA, 16 bp, Artificial Sequence
  Synthetic Oligonucleotide
agcaaatatcctcatc

SEQ ID NO: 50 - DNA, 16 bp, Artificial Sequence
  Synthetic Oligonucleotide
cagcaaatatcctcat

SEQ ID NO: 51 - DNA, 16 bp, Artificial Sequence
  Synthetic Oligonucleotide
acagcaaatatcctca

SEQ ID NO: 52 - DNA, 16 bp, Artificial Sequence
  Synthetic Oligonucleotide
gacagcaaatatcctc

SEQ ID NO: 53 - DNA, 16 bp, Artificial Sequence
  Synthetic Oligonucleotide
ctcaaaataaatagga

SEQ ID NO: 54 - DNA, 16 bp, Artificial Sequence
  Synthetic Oligonucleotide
actcaaaataaatagg

SEQ ID NO: 55 - DNA, 16 bp, Artificial Sequence
  Synthetic Oligonucleotide
gactcaaaataaatag

SEQ ID NO: 56 - DNA, 16 bp, Artificial Sequence
  Synthetic Oligonucleotide
agactcaaaataaata

SEQ ID NO: 57 - DNA, 16 bp, Artificial Sequence
  Synthetic Oligonucleotide
cagactcaaaataaat

SEQ ID NO: 58 - DNA, 16 bp, Artificial Sequence
  Synthetic Oligonucleotide
acagactcaaaataaa

SEQ ID NO: 59 - DNA, 16 bp, Artificial Sequence
  Synthetic Oligonucleotide
cacagactcaaaataa

SEQ ID NO: 60 - DNA, 16 bp, Artificial Sequence
  Synthetic Oligonucleotide
tcacagactcaaaata

SEQ ID NO: 61 - DNA, 16 bp, Artificial Sequence
  Synthetic Oligonucleotide
ctcacagactcaaaat

SEQ ID NO: 62 - DNA, 16 bp, Artificial Sequence
  Synthetic Oligonucleotide
tattaaattaatgcag

SEQ ID NO: 63 - DNA, 16 bp, Artificial Sequence
  Synthetic Oligonucleotide
ttattaaattaatgca

SEQ ID NO: 64 - DNA, 16 bp, Artificial Sequence
  Synthetic Oligonucleotide
tttattaaattaatgc

SEQ ID NO: 65 - DNA, 16 bp, Artificial Sequence
  Synthetic Oligonucleotide
ttttattaaattaatg

SEQ ID NO: 66 - DNA, 16 bp, Artificial Sequence
  Synthetic Oligonucleotide
ctgtgatctgaagtgc

SEQ ID NO: 67 - DNA, 16 bp, Artificial Sequence
  Synthetic Oligonucleotide
tctgtgatctgaagtg

SEQ ID NO: 68 - DNA, 16 bp, Artificial Sequence
  Synthetic Oligonucleotide
catctgtgatctgaag

SEQ ID NO: 69 - DNA, 16 bp, Artificial Sequence
  Synthetic Oligonucleotide
acatctgtgatctgaa

SEQ ID NO: 70 - DNA, 16 bp, Artificial Sequence
  Synthetic Oligonucleotide
ttcgcttgtagtcggc

SEQ ID NO: 71 - DNA, 16 bp, Artificial Sequence
  Synthetic Oligonucleotide
attcgcttgtagtcgg

SEQ ID NO: 72 - DNA, 16 bp, Artificial Sequence
  Synthetic Oligonucleotide
taattcgcttgtagtc

SEQ ID NO: 73 - DNA, 16 bp, Artificial Sequence
  Synthetic Oligonucleotide
gtaattcgcttgtagt

SEQ ID NO: 74 - DNA, 16 bp, Artificial Sequence
  Synthetic Oligonucleotide
gggcattgactttcac

SEQ ID NO: 75 - DNA, 16 bp, Artificial Sequence
  Synthetic Oligonucleotide
ggggcattgactttca

SEQ ID NO: 76 - DNA, 16 bp, Artificial Sequence
  Synthetic Oligonucleotide
tggggcattgactttc

SEQ ID NO: 77 - DNA, 16 bp, Artificial Sequence
  Synthetic Oligonucleotide
atggggcattgacttt

SEQ ID NO: 78 - DNA, 16 bp, Artificial Sequence
  Synthetic Oligonucleotide
tatggggcattgactt

SEQ ID NO: 79 - DNA, 16 bp, Artificial Sequence
  Synthetic Oligonucleotide
gtatggggcattgact

SEQ ID NO: 80 - DNA, 16 bp, Artificial Sequence
  Synthetic Oligonucleotide
tgtatggggcattgac

SEQ ID NO: 81 - DNA, 16 bp, Artificial Sequence
  Synthetic Oligonucleotide
ctggtcacattgaaaa

SEQ ID NO: 82 - DNA, 16 bp, Artificial Sequence
  Synthetic Oligonucleotide
gctggtcacattgaaa

SEQ ID NO: 83 - DNA, 16 bp, Artificial Sequence
  Synthetic Oligonucleotide
tgctggtcacattgaa

SEQ ID NO: 84 - DNA, 16 bp, Artificial Sequence
  Synthetic Oligonucleotide
gtgctggtcacattga

SEQ ID NO: 85 - DNA, 16 bp, Artificial Sequence
  Synthetic Oligonucleotide
agttctgggatgacca

SEQ ID NO: 86 - DNA, 16 bp, Artificial Sequence
  Synthetic Oligonucleotide
ggtagttctgggatga

SEQ ID NO: 87 - DNA, 17 bp, Artificial Sequence
  Synthetic Oligonucleotide
gcaaatatcctcatctt

SEQ ID NO: 88 - DNA, 17 bp, Artificial Sequence
  Synthetic Oligonucleotide
agcaaatatcctcatct

SEQ ID NO: 89 - DNA, 17 bp, Artificial Sequence
  Synthetic Oligonucleotide
cagcaaatatcctcatc

SEQ ID NO: 90 - DNA, 17 bp, Artificial Sequence
  Synthetic Oligonucleotide
acagcaaatatcctcat

SEQ ID NO: 91 - DNA, 17 bp, Artificial Sequence
  Synthetic Oligonucleotide
gacagcaaatatcctca

SEQ ID NO: 92 - DNA, 17 bp, Artificial Sequence
  Synthetic Oligonucleotide
actcaaaataaatagga

SEQ ID NO: 93 - DNA, 17 bp, Artificial Sequence
  Synthetic Oligonucleotide
actcaaaataaatagga

SEQ ID NO: 94 - DNA, 17 bp, Artificial Sequence
  Synthetic Oligonucleotide
agactcaaaataaatag

SEQ ID NO: 95 - DNA, 17 bp, Artificial Sequence
  Synthetic Oligonucleotide
cagactcaaaataaata

SEQ ID NO: 96 - DNA, 17 bp, Artificial Sequence
  Synthetic Oligonucleotide
acagactcaaaataaat

SEQ ID NO: 97 - DNA, 17 bp, Artificial Sequence
  Synthetic Oligonucleotide
cacagactcaaaataaa

SEQ ID NO: 98 - DNA, 17 bp, Artificial Sequence
  Synthetic Oligonucleotide
tcacagactcaaaataa

SEQ ID NO: 99 - DNA, 17 bp, Artificial Sequence
  Synthetic Oligonucleotide
ctcacagactcaaaata

SEQ ID NO: 100 - DNA, 17 bp, Artificial Sequence
  Synthetic Oligonucleotide
ttattaaattaatgcag

SEQ ID NO: 101 - DNA, 17 bp, Artificial Sequence
  Synthetic Oligonucleotide
tttattaaattaatgca

SEQ ID NO: 102 - DNA, 17 bp, Artificial Sequence
  Synthetic Oligonucleotide
ttttattaaattaatgc

SEQ ID NO: 103 - DNA, 17 bp, Artificial Sequence
  Synthetic Oligonucleotide
tctgtgatctgaagtgc

SEQ ID NO: 104 - DNA, 17 bp, Artificial Sequence
  Synthetic Oligonucleotide
acatctgtgatctgaag

SEQ ID NO: 105 - DNA, 17 bp, Artificial Sequence
  Synthetic Oligonucleotide
attcgcttgtagtcggc

SEQ ID NO: 106 - DNA, 17 bp, Artificial Sequence
  Synthetic Oligonucleotide
gtaattcgcttgtagtc

SEQ ID NO: 107 - DNA, 17 bp, Artificial Sequence
  Synthetic Oligonucleotide
ggggcattgactttcac

SEQ ID NO: 108 - DNA, 17 bp, Artificial Sequence
  Synthetic Oligonucleotide
tggggcattgactttca

SEQ ID NO: 109 - DNA, 17 bp, Artificial Sequence
  Synthetic Oligonucleotide
atggggcattgactttc

SEQ ID NO: 110 - DNA, 17 bp, Artificial Sequence
  Synthetic Oligonucleotide
tatggggcattgacttt

SEQ ID NO: 111 - DNA, 17 bp, Artificial Sequence
  Synthetic Oligonucleotide
gtatggggcattgactt

SEQ ID NO: 112 - DNA, 17 bp, Artificial Sequence
  Synthetic Oligonucleotide
tgtatggggcattgact

SEQ ID NO: 113 - DNA, 17 bp, Artificial Sequence
  Synthetic Oligonucleotide
gctggtcacattgaaaa

SEQ ID NO: 114 - DNA, 17 bp, Artificial Sequence
  Synthetic Oligonucleotide
tgctggtcacattgaaa

SEQ ID NO: 115 - DNA, 18 bp, Artificial Sequence
  Synthetic Oligonucleotide
gcaaatatcctcatcttt

SEQ ID NO: 116 - DNA, 18 bp, Artificial Sequence
  Synthetic Oligonucleotide
agcaaatatcctcatctt

SEQ ID NO: 117 - DNA, 18 bp, Artificial Sequence
  Synthetic Oligonucleotide
cagcaaatatcctcatct

SEQ ID NO: 118 - DNA, 18 bp, Artificial Sequence
  Synthetic Oligonucleotide
acagcaaatatcctcatc

SEQ ID NO: 119 - DNA, 18 bp, Artificial Sequence
  Synthetic Oligonucleotide
gacagcaaatatcctcat

SEQ ID NO: 120 - DNA, 18 bp, Artificial Sequence
  Synthetic Oligonucleotide
agacagcaaatatcctca

SEQ ID NO: 121 - DNA, 18 bp, Artificial Sequence
  Synthetic Oligonucleotide
aagacagcaaatatcctc

SEQ ID NO: 122 - DNA, 18 bp, Artificial Sequence
  Synthetic Oligonucleotide
actcaaaataaataggaa

SEQ ID NO: 123 - DNA, 18 bp, Artificial Sequence
  Synthetic Oligonucleotide
gactcaaaataaatagga

SEQ ID NO: 124 - DNA, 18 bp, Artificial Sequence
  Synthetic Oligonucleotide
agactcaaaataaatagg

SEQ ID NO: 125 - DNA, 18 bp, Artificial Sequence
  Synthetic Oligonucleotide
cagactcaaaataaatag

SEQ ID NO: 126 - DNA, 18 bp, Artificial Sequence
  Synthetic Oligonucleotide
acagactcaaaataaata

SEQ ID NO: 127 - DNA, 18 bp, Artificial Sequence
  Synthetic Oligonucleotide
cacagactcaaaataaat

SEQ ID NO: 128 - DNA, 18 bp, Artificial Sequence
  Synthetic Oligonucleotide
tcacagactcaaaataaa

SEQ ID NO: 129 - DNA, 18 bp, Artificial Sequence
  Synthetic Oligonucleotide
ctcacagactcaaaataa

SEQ ID NO: 130 - DNA, 18 bp, Artificial Sequence
  Synthetic Oligonucleotide
cctcacagactcaaaata

SEQ ID NO: 131 - DNA, 18 bp, Artificial Sequence
  Synthetic Oligonucleotide
acctcacagactcaaaat

SEQ ID NO: 132 - DNA, 18 bp, Artificial Sequence
  Synthetic Oligonucleotide
cagccttgacatgtggca

SEQ ID NO: 133 - DNA, 18 bp, Artificial Sequence
  Synthetic Oligonucleotide
ttattaaattaatgcagg

SEQ ID NO: 134 - DNA, 18 bp, Artificial Sequence
  Synthetic Oligonucleotide
tttattaaattaatgcag

SEQ ID NO: 135 - DNA, 18 bp, Artificial Sequence
  Synthetic Oligonucleotide
ttttattaaattaatgca

SEQ ID NO: 136 - DNA, 18 bp, Artificial Sequence
  Synthetic Oligonucleotide
attttattaaattaatgc

SEQ ID NO: 137 - DNA, 18 bp, Artificial Sequence
  Synthetic Oligonucleotide
tctgtgatctgaagtgca

SEQ ID NO: 138 - DNA, 18 bp, Artificial Sequence
  Synthetic Oligonucleotide
atctgtgatctgaagtgc

SEQ ID NO: 139 - DNA, 18 bp, Artificial Sequence
  Synthetic Oligonucleotide
catctgtgatctgaagtg

SEQ ID NO: 140 - DNA, 18 bp, Artificial Sequence
  Synthetic Oligonucleotide
acatctgtgatctgaagt

SEQ ID NO: 141 - DNA, 18 bp, Artificial Sequence
  Synthetic Oligonucleotide
cacatctgtgatctgaag

SEQ ID NO: 142 - DNA, 18 bp, Artificial Sequence
  Synthetic Oligonucleotide
attcgcttgtagtcggca

SEQ ID NO: 143 - DNA, 18 bp, Artificial Sequence
  Synthetic Oligonucleotide
aattcgcttgtagtcggc

SEQ ID NO: 144 - DNA, 18 bp, Artificial Sequence
  Synthetic Oligonucleotide
taattcgcttgtagtcgg

SEQ ID NO: 145 - DNA, 18 bp, Artificial Sequence
  Synthetic Oligonucleotide
gtaattcgcttgtagtcg

SEQ ID NO: 146 - DNA, 18 bp, Artificial Sequence
  Synthetic Oligonucleotide
agtaattcgcttgtagtc

SEQ ID NO: 147 - DNA, 18 bp, Artificial Sequence
  Synthetic Oligonucleotide
ggcattgactttcacagt

SEQ ID NO: 148 - DNA, 18 bp, Artificial Sequence
  Synthetic Oligonucleotide
gggcattgactttcacag

SEQ ID NO: 149 - DNA, 18 bp, Artificial Sequence
  Synthetic Oligonucleotide
ggggcattgactttcaca

SEQ ID NO: 150 - DNA, 18 bp, Artificial Sequence
  Synthetic Oligonucleotide
tggggcattgactttcac

SEQ ID NO: 151 - DNA, 18 bp, Artificial Sequence
  Synthetic Oligonucleotide
atggggcattgactttca

SEQ ID NO: 152 - DNA, 18 bp, Artificial Sequence
  Synthetic Oligonucleotide
tatggggcattgactttc

SEQ ID NO: 153 - DNA, 18 bp, Artificial Sequence
  Synthetic Oligonucleotide
gtatggggcattgacttt

SEQ ID NO: 154 - DNA, 18 bp, Artificial Sequence
  Synthetic Oligonucleotide
tgtatggggcattgactt

SEQ ID NO: 155 - DNA, 18 bp, Artificial Sequence
  Synthetic Oligonucleotide
ttgtatggggcattgact

SEQ ID NO: 156 - DNA, 18 bp, Artificial Sequence
  Synthetic Oligonucleotide
gttgtatggggcattgac

SEQ ID NO: 157 - DNA, 18 bp, Artificial Sequence
  Synthetic Oligonucleotide
tgttgtatggggcattga

SEQ ID NO: 158 - DNA, 18 bp, Artificial Sequence
  Synthetic Oligonucleotide
gctggtcacattgaaaag

SEQ ID NO: 159 - DNA, 18 bp, Artificial Sequence
  Synthetic Oligonucleotide
tgctggtcacattgaaaa

SEQ ID NO: 160 - DNA, 18 bp, Artificial Sequence
  Synthetic Oligonucleotide
gtgctggtcacattgaaa

SEQ ID NO: 161 - DNA, 18 bp, Artificial Sequence
  Synthetic Oligonucleotide
gtgtgctggtcacattga

SEQ ID NO: 162 - DNA, 18 bp, Artificial Sequence
  Synthetic Oligonucleotide
agtgtgctggtcacattg

SEQ ID NO: 163 - DNA, 18 bp, Artificial Sequence
  Synthetic Oligonucleotide
gtagttctgggatgacca

SEQ ID NO: 164 - DNA, 18 bp, Artificial Sequence
  Synthetic Oligonucleotide
ggtagttctgggatgacc

SEQ ID NO: 165 - DNA, 18 bp, Artificial Sequence
  Synthetic Oligonucleotide
aggtagttctgggatgac

SEQ ID NO: 166 - DNA, 19 bp, Artificial Sequence
  Synthetic Oligonucleotide
agcaaatatcctcatcttt

SEQ ID NO: 167 - DNA, 19 bp, Artificial Sequence
  Synthetic Oligonucleotide
cagcaaatatcctcatctt

SEQ ID NO: 168 - DNA, 19 bp, Artificial Sequence
  Synthetic Oligonucleotide
acagcaaatatcctcatct

SEQ ID NO: 169 - DNA, 19 bp, Artificial Sequence
  Synthetic Oligonucleotide
gacagcaaatatcctcatc

SEQ ID NO: 170 - DNA, 19 bp, Artificial Sequence
  Synthetic Oligonucleotide
agacagcaaatatcctcat

SEQ ID NO: 171 - DNA, 19 bp, Artificial Sequence
  Synthetic Oligonucleotide
aagacagcaaatatcctca

SEQ ID NO: 172 - DNA, 19 bp, Artificial Sequence
  Synthetic Oligonucleotide
gactcaaaataaataggaa

SEQ ID NO: 173 - DNA, 19 bp, Artificial Sequence
  Synthetic Oligonucleotide
agactcaaaataaatagga

SEQ ID NO: 174 - DNA, 19 bp, Artificial Sequence
  Synthetic Oligonucleotide
cagactcaaaataaatagg

SEQ ID NO: 175 - DNA, 19 bp, Artificial Sequence
  Synthetic Oligonucleotide
acagactcaaaataaatag

SEQ ID NO: 176 - DNA, 19 bp, Artificial Sequence
  Synthetic Oligonucleotide
cacagactcaaaataaata

SEQ ID NO: 177 - DNA, 19 bp, Artificial Sequence
  Synthetic Oligonucleotide
tcacagactcaaaataaat

SEQ ID NO: 178 - DNA, 19 bp, Artificial Sequence
  Synthetic Oligonucleotide
ctcacagactcaaaataaa

SEQ ID NO: 179 - DNA, 19 bp, Artificial Sequence
  Synthetic Oligonucleotide
cctcacagactcaaaataa

SEQ ID NO: 180 - DNA, 19 bp, Artificial Sequence
  Synthetic Oligonucleotide
acctcacagactcaaaata

SEQ ID NO: 181 - DNA, 19 bp, Artificial Sequence
  Synthetic Oligonucleotide
tttattaaattaatgcagg

SEQ ID NO: 182 - DNA, 19 bp, Artificial Sequence
  Synthetic Oligonucleotide
ttttattaaattaatgcag

SEQ ID NO: 183 - DNA, 19 bp, Artificial Sequence
  Synthetic Oligonucleotide
attttattaaattaatgca

SEQ ID NO: 184 - DNA, 19 bp, Artificial Sequence
  Synthetic Oligonucleotide
atctgtgatctgaagtgca

SEQ ID NO: 185 - DNA, 19 bp, Artificial Sequence
  Synthetic Oligonucleotide
catctgtgatctgaagtgc

SEQ ID NO: 186 - DNA, 19 bp, Artificial Sequence
  Synthetic Oligonucleotide
acatctgtgatctgaagtg

SEQ ID NO: 187 - DNA, 19 bp, Artificial Sequence
  Synthetic Oligonucleotide
aattcgcttgtagtcggca

SEQ ID NO: 188 - DNA, 19 bp, Artificial Sequence
  Synthetic Oligonucleotide
taattcgcttgtagtcggc

SEQ ID NO: 189 - DNA, 19 bp, Artificial Sequence
  Synthetic Oligonucleotide
gtaattcgcttgtagtcgg

SEQ ID NO: 190 - DNA, 19 bp, Artificial Sequence
  Synthetic Oligonucleotide
gggcattgactttcacagt

SEQ ID NO: 191 - DNA, 19 bp, Artificial Sequence
  Synthetic Oligonucleotide
ggggcattgactttcacag

SEQ ID NO: 192 - DNA, 19 bp, Artificial Sequence
  Synthetic Oligonucleotide
tggggcattgactttcaca

SEQ ID NO: 193 - DNA, 19 bp, Artificial Sequence
  Synthetic Oligonucleotide
atggggcattgactttcac

SEQ ID NO: 194 - DNA, 19 bp, Artificial Sequence
  Synthetic Oligonucleotide
tatggggcattgactttca

SEQ ID NO: 195 - DNA, 19 bp, Artificial Sequence
  Synthetic Oligonucleotide
gtatggggcattgactttc

SEQ ID NO: 196 - DNA, 19 bp, Artificial Sequence
  Synthetic Oligonucleotide
tgtatggggcattgacttt

SEQ ID NO: 197 - DNA, 19 bp, Artificial Sequence
  Synthetic Oligonucleotide
ttgtatggggcattgactt

SEQ ID NO: 198 - DNA, 19 bp, Artificial Sequence
  Synthetic Oligonucleotide
gttgtatggggcattgact

SEQ ID NO: 199 - DNA, 19 bp, Artificial Sequence
  Synthetic Oligonucleotide
tgttgtatggggcattgac

SEQ ID NO: 200 - DNA, 19 bp, Artificial Sequence
  Synthetic Oligonucleotide
tgctggtcacattgaaaag

SEQ ID NO: 201 - DNA, 19 bp, Artificial Sequence
  Synthetic Oligonucleotide
gtgctggtcacattgaaaa

SEQ ID NO: 202 - DNA, 19 bp, Artificial Sequence
  Synthetic Oligonucleotide
gtgctggtcacattgaaaa

SEQ ID NO: 203 - DNA, 19 bp, Artificial Sequence
  Synthetic Oligonucleotide
ggtagttctgggatgacca

SEQ ID NO: 204 - DNA, 19 bp, Artificial Sequence
  Synthetic Oligonucleotide
aggtagttctgggatgacc

SEQ ID NO: 205 - DNA, 20 bp, Artificial Sequence
  Synthetic Oligonucleotide
agcaaatatcctcatctttc

SEQ ID NO: 206 - DNA, 20 bp, Artificial Sequence
  Synthetic Oligonucleotide
cagcaaatatcctcatcttt

SEQ ID NO: 207 - DNA, 20 bp, Artificial Sequence
  Synthetic Oligonucleotide
acagcaaatatcctcatctt

SEQ ID NO: 208 - DNA, 20 bp, Artificial Sequence
  Synthetic Oligonucleotide
gacagcaaatatcctcatct

SEQ ID NO: 209 - DNA, 20 bp, Artificial Sequence
  Synthetic Oligonucleotide
aagacagcaaatatcctcat

SEQ ID NO: 210 - DNA, 20 bp, Artificial Sequence
  Synthetic Oligonucleotide
aaagacagcaaatatcctca

SEQ ID NO: 211 - DNA, 20 bp, Artificial Sequence
  Synthetic Oligonucleotide
agactcaaaataaataggaa

SEQ ID NO: 212 - DNA, 20 bp, Artificial Sequence
  Synthetic Oligonucleotide
cagactcaaaataaatagga

SEQ ID NO: 213 - DNA, 20 bp, Artificial Sequence
  Synthetic Oligonucleotide
acagactcaaaataaatagg

SEQ ID NO: 214 - DNA, 20 bp, Artificial Sequence
  Synthetic Oligonucleotide
cacagactcaaaataaatag

SEQ ID NO: 215 - DNA, 20 bp, Artificial Sequence
  Synthetic Oligonucleotide
tcacagactcaaaataaata

SEQ ID NO: 216 - DNA, 20 bp, Artificial Sequence
  Synthetic Oligonucleotide
ctcacagactcaaaataaat

SEQ ID NO: 217 - DNA, 20 bp, Artificial Sequence
  Synthetic Oligonucleotide
cctcacagactcaaaataaa

SEQ ID NO: 218 - DNA, 20 bp, Artificial Sequence
  Synthetic Oligonucleotide
acctcacagactcaaaataa

SEQ ID NO: 219 - DNA, 20 bp, Artificial Sequence
  Synthetic Oligonucleotide
gacctcacagactcaaaata

SEQ ID NO: 220 - DNA, 20 bp, Artificial Sequence
  Synthetic Oligonucleotide
atctgtgatctgaagtgcag

SEQ ID NO: 221 - DNA, 20 bp, Artificial Sequence
  Synthetic Oligonucleotide
catctgtgatctgaagtgca

SEQ ID NO: 222 - DNA, 20 bp, Artificial Sequence
  Synthetic Oligonucleotide
acatctgtgatctgaagtgc

SEQ ID NO: 223 - DNA, 20 bp, Artificial Sequence
  Synthetic Oligonucleotide
aattcgcttgtagtcggcac

SEQ ID NO: 224 - DNA, 20 bp, Artificial Sequence
  Synthetic Oligonucleotide
taattcgcttgtagtcggca

SEQ ID NO: 225 - DNA, 20 bp, Artificial Sequence
  Synthetic Oligonucleotide
gtaattcgcttgtagtcggc

SEQ ID NO: 226 - DNA, 20 bp, Artificial Sequence
  Synthetic Oligonucleotide
ggggcattgactttcacagt

SEQ ID NO: 227 - DNA, 20 bp, Artificial Sequence
  Synthetic Oligonucleotide
tggggcattgactttcacag

SEQ ID NO: 228 - DNA, 20 bp, Artificial Sequence
  Synthetic Oligonucleotide
atggggcattgactttcaca

SEQ ID NO: 229 - DNA, 20 bp, Artificial Sequence
  Synthetic Oligonucleotide
tatggggcattgactttcac

SEQ ID NO: 230 - DNA, 20 bp, Artificial Sequence
  Synthetic Oligonucleotide
gtatggggcattgactttca

SEQ ID NO: 231 - DNA, 20 bp, Artificial Sequence
  Synthetic Oligonucleotide
tgtatggggcattgactttc

SEQ ID NO: 232 - DNA, 20 bp, Artificial Sequence
  Synthetic Oligonucleotide
ttgtatggggcattgacttt

SEQ ID NO: 233 - DNA, 20 bp, Artificial Sequence
  Synthetic Oligonucleotide
gttgtatggggcattgactt

SEQ ID NO: 234 - DNA, 20 bp, Artificial Sequence
  Synthetic Oligonucleotide
tgttgtatggggcattgact

SEQ ID NO: 235 - DNA, 20 bp, Artificial Sequence
  Synthetic Oligonucleotide
ttgttgtatggggcattgac

SEQ ID NO: 236 - DNA, 20 bp, Artificial Sequence
  Synthetic Oligonucleotide
tgctggtcacattgaaaagc

SEQ ID NO: 237 - DNA, 20 bp, Artificial Sequence
  Synthetic Oligonucleotide
gtgctggtcacattgaaaag

SEQ ID NO: 238 - DNA, 20 bp, Artificial Sequence
  Synthetic Oligonucleotide
cagtgtgctggtcacattga

SEQ ID NO: 239 - DNA, 20 bp, Artificial Sequence
  Synthetic Oligonucleotide
aggtagttctgggatgacca

SEQ ID NO: 240 - DNA, 16 bp, Artificial Sequence
  Synthetic Oligonucleotide
gaagctgcgcagaact

SEQ ID NO: 241 - DNA, 16 bp, Artificial Sequence
  Synthetic Oligonucleotide
aatgccagtaggtcat

SEQ ID NO: 242 - DNA, 16 bp, Artificial Sequence
  Synthetic Oligonucleotide
ccgtgacagtaaatgc

SEQ ID NO: 243 - DNA, 16 bp, Artificial Sequence
  Synthetic Oligonucleotide
ctaccacatataggtc

SEQ ID NO: 244 - DNA, 16 bp, Artificial Sequence
  Synthetic Oligonucleotide
caattgtcatattgct

SEQ ID NO: 245 - DNA, 16 bp, Artificial Sequence
  Synthetic Oligonucleotide
caattagtgcagccag

SEQ ID NO: 246 - DNA, 16 bp, Artificial Sequence
  Synthetic Oligonucleotide
ctccatgcacaaattg

SEQ ID NO: 247 - DNA, 16 bp, Artificial Sequence
  Synthetic Oligonucleotide
tatgctgaaccttcag

SEQ ID NO: 248 - DNA, 16 bp, Artificial Sequence
  Synthetic Oligonucleotide
gccgggccctctgtct

SEQ ID NO: 249 - DNA, 16 bp, Artificial Sequence
  Synthetic Oligonucleotide
cctcagcctgacatgt

SEQ ID NO: 250 - DNA, 16 bp, Artificial Sequence
  Synthetic Oligonucleotide
agatgacttcggcctt

SEQ ID NO: 251 - DNA, 16 bp, Artificial Sequence
  Synthetic Oligonucleotide
ctaatctcctaaaagt

SEQ ID NO: 252 - DNA, 16 bp, Artificial Sequence
  Synthetic Oligonucleotide
tggctcccagaattac

SEQ ID NO: 253 - DNA, 16 bp, Artificial Sequence
  Synthetic Oligonucleotide
ttcttaaacggaagat

SEQ ID NO: 254 - DNA, 16 bp, Artificial Sequence
  Synthetic Oligonucleotide
aatgtgtatcactttg

SEQ ID NO: 255 - DNA, 16 bp, Artificial Sequence
  Synthetic Oligonucleotide
caatgctggattacgt

SEQ ID NO: 256 - DNA, 16 bp, Artificial Sequence
  Synthetic Oligonucleotide
ctcttgtcacgctcag

SEQ ID NO: 257 - DNA, 16 bp, Artificial Sequence
  Synthetic Oligonucleotide
caggctccctgtttga

SEQ ID NO: 258 - DNA, 16 bp, Artificial Sequence
  Synthetic Oligonucleotide
tttgaaagtatcaagg

SEQ ID NO: 259 - DNA, 16 bp, Artificial Sequence
  Synthetic Oligonucleotide
aggcgtcgatgagccc

SEQ ID NO: 260 - DNA, 16 bp, Artificial Sequence
  Synthetic Oligonucleotide
agaagtatcctttctc

SEQ ID NO: 261 - DNA, 16 bp, Artificial Sequence
  Synthetic Oligonucleotide
gatttgcttggaggct

SEQ ID NO: 262 - DNA, 16 bp, Artificial Sequence
  Synthetic Oligonucleotide
cctcaaattagggatt

SEQ ID NO: 263 - DNA, 16 bp, Artificial Sequence
  Synthetic Oligonucleotide
gagactctcagtcatg

SEQ ID NO: 264 - DNA, 16 bp, Artificial Sequence
  Synthetic Oligonucleotide
taaatactgtcccgtt

SEQ ID NO: 265 - DNA, 16 bp, Artificial Sequence
  Synthetic Oligonucleotide
tctactacaatatatc

SEQ ID NO: 266 - DNA, 16 bp, Artificial Sequence
  Synthetic Oligonucleotide
tagtttggcgacaaaa

SEQ ID NO: 267 - DNA, 16 bp, Artificial Sequence
  Synthetic Oligonucleotide
gagcaaatcattaagc

SEQ ID NO: 268 - DNA, 16 bp, Artificial Sequence
  Synthetic Oligonucleotide
atagaggagaccaagc

SEQ ID NO: 269 - DNA, 16 bp, Artificial Sequence
  Synthetic Oligonucleotide
atgcaaccaacggttt

SEQ ID NO: 270 - DNA, 16 bp, Artificial Sequence
  Synthetic Oligonucleotide
agattaggtcaaccag

SEQ ID NO: 271 - DNA, 16 bp, Artificial Sequence
  Synthetic Oligonucleotide
ggctaccacataattg

SEQ ID NO: 272 - DNA, 16 bp, Artificial Sequence
  Synthetic Oligonucleotide
cgatgaaatgagatta

SEQ ID NO: 273 - DNA, 16 bp, Artificial Sequence
  Synthetic Oligonucleotide
gttatcacaacagggt

SEQ ID NO: 274 - DNA, 16 bp, Artificial Sequence
  Synthetic Oligonucleotide
tgtacgatgggtaaaa

SEQ ID NO: 275 - DNA, 16 bp, Artificial Sequence
  Synthetic Oligonucleotide
agctgtaaattgtatt

SEQ ID NO: 276 - DNA, 16 bp, Artificial Sequence
  Synthetic Oligonucleotide
gcacagcgattgatat

SEQ ID NO: 277 - DNA, 16 bp, Artificial Sequence
  Synthetic Oligonucleotide
tctcctcattatacta

SEQ ID NO: 278 - DNA, 16 bp, Artificial Sequence
  Synthetic Oligonucleotide
ttatgctatgacactg

SEQ ID NO: 279 - DNA, 16 bp, Artificial Sequence
  Synthetic Oligonucleotide
tcgggttttcccctcg

SEQ ID NO: 280 - DNA, 16 bp, Artificial Sequence
  Synthetic Oligonucleotide
aaccgtcccagaccac

SEQ ID NO: 281 - DNA, 16 bp, Artificial Sequence
  Synthetic Oligonucleotide
gggttattttaagtac

SEQ ID NO: 282 - DNA, 16 bp, Artificial Sequence
  Synthetic Oligonucleotide
attagattatatggca

SEQ ID NO: 283 - DNA, 16 bp, Artificial Sequence
  Synthetic Oligonucleotide
aatgaaaggcagtagc

SEQ ID NO: 284 - DNA, 16 bp, Artificial Sequence
  Synthetic Oligonucleotide
ctatgggaaagataac

SEQ ID NO: 285 - DNA, 16 bp, Artificial Sequence
  Synthetic Oligonucleotide
gtaggacatatttaac

SEQ ID NO: 286 - DNA, 16 bp, Artificial Sequence
  Synthetic Oligonucleotide
gtaggacatatttaac

SEQ ID NO: 287 - DNA, 16 bp, Artificial Sequence
  Synthetic Oligonucleotide
ctgtcctagagcaaat

SEQ ID NO: 288 - DNA, 16 bp, Artificial Sequence
  Synthetic Oligonucleotide
gactagattgactcag

SEQ ID NO: 289 - DNA, 16 bp, Artificial Sequence
  Synthetic Oligonucleotide
gttaataataagattg

SEQ ID NO: 290 - DNA, 16 bp, Artificial Sequence
  Synthetic Oligonucleotide
agcatcaaagtgaagt

SEQ ID NO: 291 - DNA, 16 bp, Artificial Sequence
  Synthetic Oligonucleotide
aggtacactgccggaa

SEQ ID NO: 292 - DNA, 16 bp, Artificial Sequence
  Synthetic Oligonucleotide
tcaagcacaacgaatg

SEQ ID NO: 293 - DNA, 16 bp, Artificial Sequence
  Synthetic Oligonucleotide
tgacagctggtggcat

SEQ ID NO: 294 - DNA, 16 bp, Artificial Sequence
  Synthetic Oligonucleotide
cctcttaggagggctg

SEQ ID NO: 295 - DNA, 16 bp, Artificial Sequence
  Synthetic Oligonucleotide
ctgaatctcgaaacct

SEQ ID NO: 296 - DNA, 16 bp, Artificial Sequence
  Synthetic Oligonucleotide
gagctctgttggagac

SEQ ID NO: 297 - DNA, 16 bp, Artificial Sequence
  Synthetic Oligonucleotide
tcacaccaattactgt

SEQ ID NO: 298 - DNA, 16 bp, Artificial Sequence
  Synthetic Oligonucleotide
aggaatagactgagta

SEQ ID NO: 299 - DNA, 16 bp, Artificial Sequence
  Synthetic Oligonucleotide
ggataaagtgccttac

SEQ ID NO: 300 - DNA, 16 bp, Artificial Sequence
  Synthetic Oligonucleotide
ttacgatgaaacatga

SEQ ID NO: 301 - DNA, 16 bp, Artificial Sequence
  Synthetic Oligonucleotide
agaaaatggacatgct